(19) United States
(12) Patent Application Publication
(10) Pub. No.: US 2023/0248386 A1
(43) Pub. Date: Aug. 10, 2023

(54) METHODS OF TREATING VASCULAR LESIONS

(71) Applicant: BOSTON SCIENTIFIC SCIMED, INC., MAPLE GROVE, MN (US)

(72) Inventor: GORDON JOHN KOCUR, LINO LAKES, MN (US)

(73) Assignee: BOSTON SCIENTIFIC SCIMED, INC., MAPLE GROVE, MN (US)

(21) Appl. No.: 18/107,182

(22) Filed: Feb. 8, 2023

Related U.S. Application Data
(60) Provisional application No. 63/308,811, filed on Feb. 10, 2022.

(51) Int. Cl. A61B 17/3207

(52) U.S. Cl. CPC A61B 17/320725; A61M 2025/1086

(57) ABSTRACT

A catheter is advanced to a treatment site proximate a lesion, the catheter including a plurality of traction elements and an inflatable balloon adapted to urge the plurality of traction elements radially outwardly when the inflatable balloon is inflated. The inflatable balloon is inflated to urge the plurality of traction elements radially outwardly into contact with the lesion with the distal region at a first rotational orientation. The inflatable balloon is deflated and the proximal region of the catheter is rotated in order to rotate the distal region to a second rotational orientation different from the first rotational orientation. The inflatable balloon is inflated to again urge the plurality of traction elements radially outwardly into contact with the lesion. The catheter is adapted to provide a substantially one-to-one rotational arrangement between the proximal region and the distal region.

### CROSS-REFERENCE TO RELATED APPLICATIONS

This application claims the benefit of U.S. Provisional Pat. Application Serial No. 63/308,811, filed Feb. 10, 2022, which is incorporated herein by reference.

### TECHNICAL FIELD

The present disclosure pertains to medical devices, and methods for manufacturing and using medical devices. More particularly, the disclosure is directed to devices and associated methods for treating intravascular lesions.

### BACKGROUND

Coronary and peripheral artery stenosis is primarily due to deposits of cholesterol, calcium and fibrotic tissue, with the fibrotic tissue typically being the dominant of the three components and calcium being the most resistant to dilation with a balloon. It happens that a large proportion of stenoses are formed as eccentric lesions (i.e. lesions that do not extend completely around the circumference of the affected body vessel). A suitable remedy would effectively treat an eccentric stenosis without adversely affecting healthy, non-diseased tissue. Dilation of stenoses using standard angioplasty balloons has enjoyed widespread acceptance in the treatment of stenoses, however, this treatment protocol suffers from a high rate of acute vascular recoil and restenosis. Recent studies, however, indicate that the rate of acute vascular recoil can be reduced if the stenosis that is being dilated is also incised. With incision, some stenoses can be more easily flattened at lower pressures, and the likelihood of damaging the artery during dilation may be reduced. The circumferential location of incisions relative to an eccentric lesion can impact the success of treatment. A need remains for methods and devices for treating eccentric lesions.

### SUMMARY

This disclosure provides design, material, manufacturing method, and use alternatives for medical devices. As an example, a device for treating an eccentric lesion within a blood vessel is disclosed. The device comprises an elongate shaft including a distal region and a proximal region. The elongate shaft is configured to provide torque transmission between the proximal region and the distal region. A hub is fixedly secured to the proximal region of the elongate shaft, and an inflatable balloon fixedly secured to the distal region of the elongate shaft. A plurality of traction elements are disposed on the inflatable balloon such that the plurality of traction elements are urged radially outwardly when the inflatable balloon is inflated such that at least two of the plurality of traction elements concurrently engage the eccentric lesion within the blood vessel.

Alternatively or additionally, the elongate shaft may be configured to provide, for a given angle of rotation of the hub, a rotation of the balloon in the same direction an angle of rotation that is within twenty percent of the given angle of rotation of the hub.

Alternatively or additionally, the elongate shaft may be configured to provide, for a given angle of rotation of the hub, a rotation of the balloon in the same direction an angle of rotation that is within ten percent of the given angle of rotation of the hub.

Alternatively or additionally, the elongate shaft may include one or more torque-transmission elements extending within the elongate shaft.

Alternatively or additionally, the one or more torque-transmission elements may include a reinforcing member extending within a tubular member of the elongate shaft.

Alternatively or additionally, the reinforcing member may include at least one of a braid and a coil.

Alternatively or additionally, the braid or coil may extend from the hub to a proximal waist of the balloon.

Alternatively or additionally, the braid or coil may extend from the hub, through the balloon to a distal waist of the balloon.

Alternatively or additionally, the plurality of traction elements may be configured such that urging the plurality of traction elements radially outwardly into contact with the lesion causes at least a portion of the lesion to stretch or crack.

Alternatively or additionally, the plurality of traction elements may be a plurality of cutting members circumferentially spaced around an outer surface of the inflatable balloon.

Alternatively or additionally, the plurality of traction elements may together form a wire cage disposed about the inflatable balloon.

Alternatively or additionally, the plurality of traction elements may include a plurality of protuberances formed on an outer surface of the balloon.

Another example is a method of treating a lesion in a blood vessel. The method includes advancing a catheter through the vessel to a treatment site proximate the lesion. The catheter includes a torqueable shaft extending from a hub fixedly secured to a proximal end region of the torqueable shaft to an expandable lesion-engaging portion fixedly secured to a distal end region of the torqueable shaft. The expandable lesion-engaging portion includes an inflatable balloon and a plurality of traction elements arranged around the balloon. Thereafter, the expandable lesion-engaging portion is expanded radially outward to engage the lesion. Thereafter, radially contracting the expandable lesion-engaging portion is radially contracted. Thereafter, the hub is rotated a desired rotational angle to thereby rotate the expandable lesion engaging portion a corresponding amount. Thereafter, the expandable lesion-engaging portion is re-expanded radially outward to engage the lesion.

Alternatively or additionally, the lesion may be an eccentric lesion extending less than 180 degrees around the vessel, wherein during the step of expanding the expandable lesion-engaging portion and/or the step of re-expanding the expandable lesion-engaging portion, at least two of the plurality of traction elements concurrently engage the eccentric lesion to stretch or crack the lesion.

Alternatively or additionally, the method may further include translating the catheter longitudinally within the vessel before re-expanding the expandable lesion-engaging portion.

Another example is a method of treating a lesion within a blood vessel. The method comprises advancing a catheter through the vessel to a treatment site proximate the lesion. The catheter includes a torqueable shaft extending from a hub fixedly secured to a proximal end region of the torqueable shaft to a balloon fixedly secured to a distal end region of the torqueable shaft. The catheter includes a plurality of traction elements disposed about the inflatable balloon. The inflatable balloon is adapted to urge the plurality of traction elements radially outwardly when the inflatable balloon is inflated. Thereafter, the inflatable balloon is inflated to urge one or more of the plurality of traction elements radially outwardly into contact with the lesion. Thereafter, the inflatable balloon is deflated. Thereafter, the hub of the catheter is rotated in order to achieve a corresponding rotation of the balloon of the catheter relative to the treatment site. Thereafter, the inflatable balloon is re-inflated to urge one or more of the plurality of traction elements radially outwardly into contact with the lesion. At least two of the plurality of traction elements concurrently engage the lesion during the step of inflating the inflatable balloon and/or during the step of re-inflating the inflatable balloon.

Alternatively or additionally, for a given angle of rotation of the hub of the catheter, the balloon of the catheter may rotate in the same direction an angle or rotation that is within twenty percent of the given angle of rotation.

Alternatively or additionally, for a given angle of rotation of the hub of the catheter, the balloon of the catheter may rotate in the same direction an angle of rotation that is within ten percent of the given angle of rotation.

Alternatively or additionally, urging the at least two of the plurality of traction elements radially outwardly into contact with the lesion may cause at least a portion of the lesion to stretch or crack.

Alternatively or additionally, the plurality of traction elements may be a plurality of cutting members circumferentially spaced around an outer surface of the inflatable balloon.

Alternatively or additionally, the torqueable shaft may include a braid or coil extending from the hub to a proximal waist of the balloon.

Alternatively or additionally, the lesion may be an eccentric lesion extending less than 180 degrees around the vessel, wherein the lesion is cracked during the step of inflating the inflatable balloon and/or the step of re-inflating the inflatable balloon.

Another example is a method of treating a lesion within a blood vessel. The method comprises advancing a catheter to a treatment site proximate a lesion. The catheter includes a proximal region including a hub and a distal region including a plurality of traction elements and an inflatable balloon adapted to urge the plurality of traction elements radially outwardly when the inflatable balloon is inflated. Thereafter, the inflatable balloon is inflated to urge at least one of the plurality of traction elements radially outwardly into contact with the lesion with the distal region at a first rotational orientation. Thereafter, the inflatable balloon is deflated. Thereafter, the proximal region of the catheter is rotated in order to rotate the distal region to a second rotational orientation different from the first rotational orientation. Thereafter, the inflatable balloon is re-inflated to urge at least one of the plurality of traction elements radially outwardly into contact with the lesion. The catheter is adapted to provide a substantially one-to-one rotational arrangement between the proximal region and the distal region. At least two of the plurality of traction elements concurrently engage the lesion during the step of inflating the inflatable balloon and/or during the step of re-inflating the inflatable balloon.

Alternatively or additionally, rotating the proximal region of the catheter a given angle of rotation may result in rotation of the distal region that is within twenty percent of the given angle of rotation.

Alternatively or additionally, rotating the proximal region of the catheter a given angle of rotation may result in rotation of the distal region that is within ten percent of the given angle of rotation.

Alternatively or additionally, the catheter may include a torqueable shaft extending from the hub to the inflatable balloon, the torqueable shaft including a braid or coil extending from the hub to a proximal waist of the balloon.

Alternatively or additionally, the lesion may be an eccentric lesion extending less than 180 degrees around the vessel, wherein the lesion is cracked during the step of inflating the inflatable balloon and/or the step of re-inflating the inflatable balloon.

As another example, a device for treating a lesion within a blood vessel includes an elongate shaft including a distal region and a proximal region, the elongate shaft configured to provide torque transmission between the proximal region and the distal region. An inflatable balloon is disposed on the distal region of the elongate shaft. A plurality of traction elements are disposed on the inflatable balloon such that the plurality of traction elements are urged radially outwardly when the inflatable balloon is inflated.

Alternatively or additionally, the elongate shaft may be configured to provide, for a given rotation of the proximal region of the elongate shaft, a rotation of the distal region of the elongate shaft in the same direction a rotational distance that is within twenty percent of the given rotation.

Alternatively or additionally, the elongate shaft may be configured to provide, for a given rotation of the proximal region of the elongate shaft, a rotation of the distal region of the elongate shaft in the same direction a rotational distance that is within ten percent of the given rotation.

Alternatively or additionally, the elongate shaft may include one or more torque-transmission elements extending within the elongate shaft.

Alternatively or additionally, the elongate shaft may include an inner tubular member defining a guidewire lumen and an outer tubular member, with a space between the inner tubular member and the outer tubular member defining an inflation lumen, and a reinforcing member extending within at least one of the inner tubular member and the outer tubular member.

Alternatively or additionally, the reinforcing member may include at least one of a braid and a coil.

Alternatively or additionally, the elongate shaft may define a guidewire lumen extending through at least a portion of the elongate shaft.

Alternatively or additionally, the elongate shaft may define an inflation lumen extending through the elongate shaft, the inflation lumen fluidly coupled with an interior of the inflatable balloon.

Alternatively or additionally, the traction elements may be configured such that urging the plurality of traction elements radially outwardly into contact with the lesion causes at least a portion of the lesion to stretch or crack.

Alternatively or additionally, the plurality of traction elements may be oriented in an axial direction and are circumferentially spaced on an outer surface of the inflatable balloon.

Alternatively or additionally, the plurality of traction elements may together form a wire cage disposed about the inflatable balloon.

Alternatively or additionally, the plurality of traction elements may include an outer surface of the balloon.

As another example, a method of treating a lesion within a blood vessel includes advancing a catheter to a treatment site proximate a lesion, the catheter including a proximal region and a distal region, the distal region including a plurality of traction elements and an inflatable balloon adapted to urge the plurality of traction elements radially outwardly when the inflatable balloon is inflated. The inflatable balloon is inflated to urge the plurality of traction elements radially outwardly into contact with the lesion. The inflatable balloon is deflated and the proximal region of the catheter is rotated in order to achieve a corresponding rotation of the distal region of the catheter relative to the treatment site. The inflatable balloon is inflated again to urge the plurality of traction elements radially outwardly into contact with the lesion.

Alternatively or additionally, for a given rotation of the proximal region of the catheter, the distal region of the catheter may rotate in the same direction a rotational distance that is within twenty percent of the given rotation.

Alternatively or additionally, for a given rotation of the proximal region of the catheter, the distal region of the catheter may rotate in the same direction a rotational distance that is within ten percent of the given rotation.

Alternatively or additionally, urging the plurality of traction elements radially outwardly into contact with the lesion may cause at least a portion of the lesion to stretch or crack.

Alternatively or additionally, the plurality of traction elements may be oriented in an axial direction and may be circumferentially spaced on an outer surface of the inflatable balloon.

Alternatively or additionally, the plurality of traction elements together may form a wire cage disposed about the inflatable balloon.

Alternatively or additionally, the plurality of traction elements may include an outer surface of the balloon.

Alternatively or additionally, the catheter may include an elongate shaft and one or more torque-transmission elements extending within the elongate shaft.

As another example, a method of treating a lesion within a blood vessel includes advancing a catheter to a treatment site proximate a lesion, the catheter including a proximal region and a distal region, the distal region including a plurality of traction elements and an inflatable balloon adapted to urge the plurality of traction elements radially outwardly when the inflatable balloon is inflated. The inflatable balloon is inflated to urge the plurality of traction elements radially outwardly into contact with the lesion with the distal region at a first rotational orientation. The inflatable balloon is deflated and the proximal region of the catheter is rotated in order to rotate the distal region to a second rotational orientation different from the first rotational orientation. The inflatable balloon is inflated to again urge the plurality of traction elements radially outwardly into contact with the lesion. The catheter is adapted to provide a substantially one-to-one rotational arrangement between the proximal region and the distal region.

Alternatively or additionally, rotating the proximal region of the catheter a given rotational distance may result in a rotation of the distal region that is within twenty percent of the given rotational distance.

Alternatively or additionally, rotating the proximal region of the catheter a given rotational distance may result in a rotation of the distal region that is within ten percent of the given rotational distance.

Alternatively or additionally, the method may further include repeatedly deflating the inflatable balloon, rotating the proximal region of the catheter such that the distal region of the catheter gains a new rotational orientation, and inflating the inflatable balloon to urge the plurality of traction elements radially outwardly into contact with the lesion with the distal region at each of a plurality of rotational orientations in order to increase the chances of successfully stretching and cracking the lesion by engaging the lesion with two of the plurality of traction elements.

Alternatively or additionally, the plurality of traction elements may be oriented in an axial direction and may be circumferentially spaced on an outer surface of the inflatable balloon.

Alternatively or additionally, the plurality of traction elements together may form a wire cage disposed about the inflatable balloon.

Alternatively or additionally, the plurality of traction elements may include an outer surface of the balloon.

As another example, a method of treating a lesion includes advancing a catheter through a vessel to a treatment site proximate a lesion, the catheter including an expandable lesion-engaging portion and a torqueable shaft extending proximally from the expandable lesion-engaging portion. The expandable lesion-engaging portion is expanded to engage and stretch the lesion. The expandable lesion-engaging portion is contracted and the expandable lesion-engaging portion is rotated a desired rotational distance by rotating the torqueable shaft. The expandable lesion-engaging portion is expanded again to engage and stretch the lesion.

Alternatively or additionally, the method may further include repeatedly contracting the expandable lesion-engaging portion, rotating the expandable lesion-engaging portion a desired rotational distance by rotating the torqueable shaft, and expanding the expandable lesion-engaging portion to engage and stretch the lesion at each of a plurality of rotational orientations in order to increase the chances of successfully stretching and cracking the lesion by engaging the lesion with two of the plurality of traction elements.

Alternatively or additionally, the expandable lesion-engaging portion may include a plurality of traction elements and an inflatable balloon adapted to urge the plurality of traction elements radially outwardly when the inflatable balloon is inflated.

Alternatively or additionally, the method may further include translating the catheter within the vessel before expanding the expandable lesion-engaging portion again.

Alternatively or additionally, rotating the distal region a desired rotation distance may include rotating the torqueable shaft a corresponding rotational distance.

As another example, a method of treating a lesion within a blood vessel includes advancing a catheter to a treatment site proximate a lesion, the catheter including a proximal region including a hub and a distal region including a plurality of traction elements and an inflatable balloon adapted to urge the plurality of traction elements radially outwardly when the inflatable balloon is inflated, the catheter including a mechanism disposed between the proximal region and the distal region, the mechanism adapted to convert axial movement of the proximal region into rotation of the distal region. The inflatable balloon is inflated to urge at least one of the plurality of traction elements radially outwardly into contact with the lesion with the distal region at a first rotational orientation. The proximal region is moved axially, thereby causing the mechanism to twist the distal region, temporarily placing energy into the distal region. The inflatable balloon is deflated in order to allow the distal region to untwist, thereby rotating the distal region to a second rotational orientation different form the first rotational orientation. The inflatable balloon is re-inflated to urge at least one of the plurality of traction elements radially outwardly into contact with the lesion. At least two of the plurality of traction elements concurrently engage the lesion during the step of inflating the inflatable balloon and/or during the step of re-inflating the inflatable balloon.

Alternatively or additionally, the mechanism may be adapted such that pushing on the proximal region of the catheter causes the mechanism to twist the distal region.

Alternatively or additionally, the mechanism is adapted such that pulling on the proximal region of the catheter causes the mechanism to twist the distal region.

Alternatively or additionally, the catheter may include a shaft extending from the hub to the inflatable balloon.

Alternatively or additionally, the lesion is an eccentric lesion extending less than 180 degrees around the vessel, wherein the lesion is cracked during the step of inflating the inflatable balloon and/or the step of re-inflating the inflatable balloon.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic side view of an illustrative catheter usable for treating vascular lesions;

FIG. 1A is a cross-sectional view taken along line 1A-1A of FIG. 1;

FIG. 2 is a schematic cross-sectional view showing an elongate shaft that may be used to form the illustrative catheter of FIG. 1;

FIG. 3 is a schematic cross-sectional view showing an elongate shaft that may be used to form the illustrative catheter of FIG. 1;

FIG. 4 is a schematic cross-sectional view of a vessel and a vascular eccentric lesion and a treatment catheter disposed within the vessel at a first rotational orientation with one traction element contacting the lesion;

FIG. 5A is a schematic cross-sectional view of a vessel and a vascular eccentric lesion and the treatment catheter of FIG. 4 disposed within the vessel at a second rotational orientation with two traction elements contacting the lesion;

FIG. 5B is a schematic cross-section view of a vessel and a vascular eccentric lesion and the treatment catheter of FIG. 5A with the expandable element, such as an inflatable balloon, further radially expanded to crack the vascular eccentric lesion;

FIG. 6 is a cross-sectional view of a vessel and a vascular eccentric lesion and a treatment catheter disposed within the vessel at a first rotational orientation with one traction element contacting the lesion;

FIG. 7A is a cross-sectional view of a vessel and a vascular eccentric lesion and the treatment catheter of FIG. 6 disposed within the vessel at a second rotational orientation with two traction elements contacting the lesion;

FIG. 7B is a schematic cross-section view of a vessel and a vascular eccentric lesion and the treatment catheter of FIG. 7A with the expandable element, such as an inflatable balloon, further radially expanded to crack the vascular eccentric lesion;

FIG. 8 is a schematic side view of an illustrative catheter usable for treating vascular lesions, the catheter shown in a deflated configuration;

FIG. 9 is a schematic side view of the illustrative catheter of FIG. 8, the catheter shown in an inflated configuration;

FIG. 10 is a schematic side view of an illustrative catheter usable for treating vascular lesions, the catheter shown in an inflated configuration;

FIG. 11 is a flow diagram showing an illustrative method of treating a vascular lesion;

FIG. 12 is a flow diagram showing an illustrative method of treating a vascular lesion;

FIG. 13 is a flow diagram showing an illustrative method of treating a vascular lesion;

FIG. 14 is a flow diagram showing an illustrative method of treating a vascular lesion;

FIG. 15 is a flow diagram showing an illustrative method of treating a vascular lesion;

FIG. 16 is a flow diagram showing an illustrative method of treating a vascular lesion;

FIGS. 17A through 17C are schematic side views of an illustrative catheter that is adapted to allow a distal region of the catheter to be rotated in response to pulling on a proximal region of the catheter;

FIGS. 18A and 18B are schematic side views of an illustrative catheter that is adapted to allow a distal region of the catheter to be rotated in response to pushing on a proximal region of the catheter;

FIGS. 18C through 18G are schematic views of an illustrative mechanism included in the illustrative catheter of FIGS. 18A and 18B;

FIGS. 19A and 19B are schematic views of an illustrative catheter that is adapted to allow a distal region of the catheter to be rotated in response to rotating a proximal region of the catheter in a first rotational direction;

FIG. 19C is a schematic view of an illustrative mechanism included in the illustrative catheter of FIGS. 19A and 19B; and

FIG. 20 is a schematic cross-sectional view of an illustrative inflatable balloon showing formation of traction elements.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Occluded, stenotic, or narrowed blood vessels, as well as native or synthetic arteriovenous dialysis fistulae, may be treated in a recanalization procedure, such as with an angioplasty balloon catheter advanced over a guidewire to an occlusion so that the balloon is positioned across the occlusion. The balloon is then inflated to enlarge the passageway through the occlusion.

One of the major obstacles in treating coronary artery disease and/or treating blocked blood vessels or fistulae is resistance to dilation with a standard angioplasty balloon and/or vascular recoil of the lesion following dilation or other recanalization procedure. Evidence has shown that cutting or scoring the stenosis can be beneficial. In some instances, it can be helpful to stretch the stenosis, or portions thereof, causing the stenosis, or portions thereof, to stretch.

Depending on the level of plaque (thickness and length) in vessels it may be difficult for a physician to expand the internal diameter of the vessel sufficiently to successfully restore blood flow. The compliance of the vessel may need to be improved such that an inflated balloon will cause an expansion in the internal diameter of the vessel that will remain after the balloon is removed.

It will be appreciated that some lesions, or stenoses, are relatively small, and extend only a relatively short distance around the circumference of a vessel. Some lesions are more extensive, and can extend a substantial distance or even all the way around the circumference of a vessel. In some cases, there may be a desire to place a treatment catheter that includes an inflatable balloon and a plurality of traction elements that are disposed relative to the inflatable balloon such that inflating the inflatable balloon can cause the plurality of traction elements to move outwardly, into or against the lesion. This can stretch the lesion circumferentially and can cause the lesion to crack, particularly if enough of the plurality of traction elements properly contact the lesion itself.

However, because lesions are of varying size, and because there isn’t a good way to ascertain the rotational orientation of the plurality of traction elements relative to the exact location of the lesion, a physician or other professional may wish to inflate the inflatable balloon at a particular rotational orientation of the plurality of traction elements, then deflate the inflatable balloon, rotate the inflatable balloon in order to change the relative orientation of the plurality of traction elements before inflating the inflatable balloon once again in order to urge the plurality of traction elements outwardly once again, with the hope that at least some of the plurality of traction elements will contact the lesion in the optimal orientation.

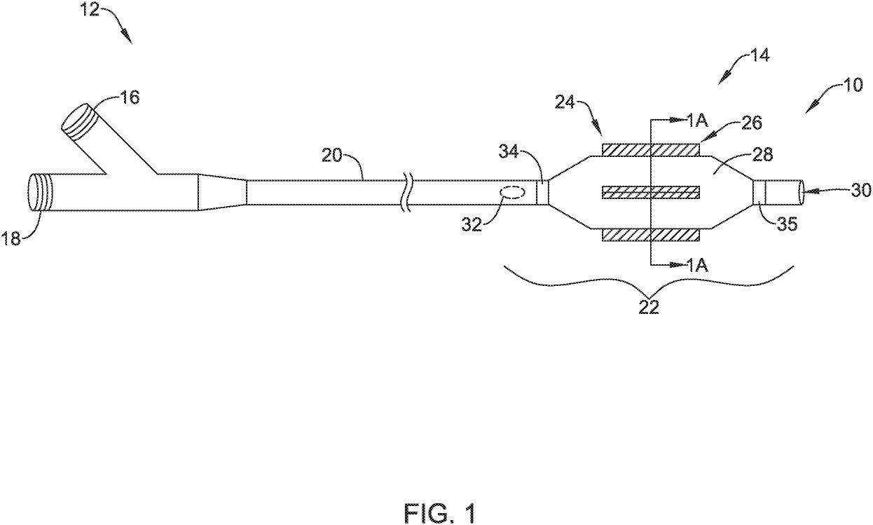

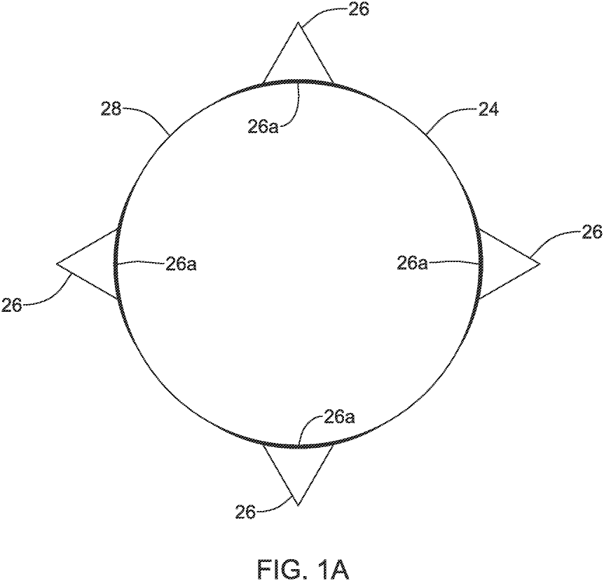

FIG. 1 is a schematic side view of an illustrative catheter 10 that may be used to treat a lesion within a blood vessel. The illustrative catheter 10 extends from a proximal region 12 to a distal region 14. In some cases, as shown, the proximal region 12 may include a hub or manifold having one or more Luer fittings 16 and 18 that may be used to gain access to one or more lumens extending through the catheter 10. The catheter 10 includes an elongate shaft 20 that extends between the proximal region 12 and the distal region 14. For example, the proximal end of the elongate shaft 20 may be fixedly secured to the hub or manifold, and the distal end of the elongate shaft 20 may be fixedly secured to an inflatable balloon of the catheter 10.

As will be discussed, the elongate shaft 20 may be adapted to provide torqueability, meaning that a particular rotation made at the proximal region 12 will be communicated to the distal region 14. In some cases, the elongate shaft 20 may be adapted to provide sufficient torqueability such that a particular rotation made at the proximal region 12 (e.g., rotation of the hub or manifold) will be communicated, within plus or minus twenty (20) percent at the distal region 14, thus rotating an inflatable balloon provided at the distal region 14 a corresponding amount. As an example, a 40-degree rotation made at the proximal region 12 will correlate to a rotation made at the distal region 14 that is in the range of 32 degrees to 48 degrees. In some cases, the elongate shaft 20 may be adapted to provide sufficient torqueability such that a particular rotation made at the proximal region 12 will be communicated, within plus or minus ten percent (±10%) at the distal region 14. As an example, a 40-degree rotation made at the proximal region 12 will correlate to a rotation made at the distal region 14 that is in the range of 36 degrees to 44 degrees. In some cases, the rotation made at the proximal region 12 will result in a rotation made at the distal region 14 that is within five percent ((±5%), or even less, of the movement at the proximal region 12. These are just examples.

The distal region 14 includes what may be considered as being an expandable lesion-engaging portion 22. The expandable lesion-engaging portion 22 is the part of the catheter 10 that is adapted to engage the lesion, hopefully stretching the lesion and causing the lesion to at least begin to crack. As shown in FIG. 1, the expandable lesion-engaging portion 22 includes an inflatable balloon 24 that is secured relative to the elongate shaft 20 as well as a plurality of traction elements 26 that are secured relative to an outer surface 28 of the inflatable balloon 24.

In some cases, the catheter 10 may be considered as being an OTW (over the wire) catheter, and thus may include a guidewire lumen 30 that extends through the proximal region 12, the elongate shaft 20 and the distal region 14. In some cases, the Luer fitting 18 may be aligned with, and provide access to, the proximal end of the guidewire lumen 30. It will be appreciated that in cases in which the catheter 10 is instead an SOE (single operator exchange) catheter, the guidewire lumen 30 would not extend all the way back through the elongate shaft 20, but would instead terminate at a proximal guidewire port 32 (shown in phantom). In either event, the guidewire lumen 30 would extend through the expandable lesion-engaging portion 22 and would terminate at the distal end of the catheter 10.

In some cases, an inflation lumen may extend through the elongate shaft 20 and may be fluidly coupled with an interior of the inflatable balloon 24. For example, the Luer fitting 16 may be adapted to be coupled to a source of inflation fluid such as saline, and may be fluidly coupled with the inflation lumen (not visible in FIG. 1).

The plurality of traction elements 26 may take a variety of different forms. In some cases, at least some of the plurality of traction elements 26 may include polymeric or metallic strips that are axially aligned along the length of the inflatable balloon 24 and are radially spaced apart around the circumference of the inflatable balloon 24. In some cases, each traction element 26 may be a single polymeric member. In some cases, each traction element 26 may include two, three or more polymeric members that are axially aligned along the length of the inflatable balloon 24. In some instances, the traction elements 26 may not be axially aligned along the length of the inflatable balloon 24, but may instead be disposed at an acute angle with respect to the length of the inflatable balloon 24. In some instances, at least some of the traction elements 26 may extend helically about the inflatable balloon 24

In some cases, at least some of the plurality of traction elements 26 may be considered as including cutting surfaces, for example, (e.g., cutting blades or atherotomes). FIG. 1A is a cross-sectional view of the inflatable balloon 24, taken along line 1A-1A of FIG. 1A. As can be seen, each of the traction elements 26 are cutting blades that are attached to the outer surface 28 of the inflatable balloon 24 via polymeric members 26a that help to anchor the traction elements 26 (cutting blades or atherotomes) to the inflatable balloon 24.

For example, if there are a total of four traction elements 26, as shown, the traction elements 26 may each be spaced circumferentially about 90 degrees apart from the adjacent traction element 26. If there are a total of three traction elements 26, each of the traction elements 26 may be spaced circumferentially about 120 degrees apart from the adjacent traction element 26. If there are a total of five traction elements 26, for example, each would be spaced circumferentially about 72 degrees. These are just examples. In some cases, the traction elements 26 may not be equally circumferentially spaced about the inflatable balloon 24. In some instances, a plurality of traction elements 26 could be arranged about the inflatable balloon 24, with each of the plurality of traction elements 26 extending at a different angle with respect to the length of the inflatable balloon 24. In some instances, a single traction element 26 may extend helically around the inflatable balloon 24, and may include several rotations around the circumference of the inflatable balloon 24.

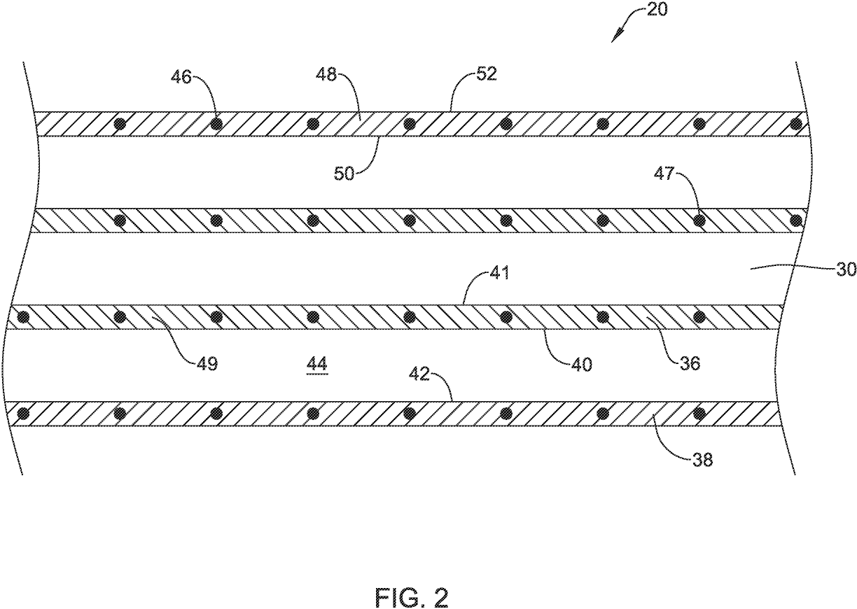

As noted, the elongate shaft 20 is adapted to provide torqueability. FIG. 2 is a schematic cross-sectional view of a portion of the elongate shaft 20. FIG. 2 shows that the elongate shaft 20, or at least the illustrated portion thereof, includes an inner shaft 36 and an outer shaft 38. As seen, the inner shaft 36 defines the guidewire lumen 30. An annular space 44 is defined between an outer surface 40 of the inner shaft 36 and an inner surface 42 of the outer shaft 38 and in some cases the annular space 44 may function as an inflation lumen. It will be appreciated that the illustrated portion of the elongate shaft 20 could correspond to essentially any portion of the elongate shaft 20 when the catheter 10 is an OTW catheter, or just distal of the proximal guidewire port 32 when the catheter 10 is an SOE catheter. For an SOE catheter, the proximal shaft will also be torque transmitting, and thus the proximal shaft of an SOE catheter may include a tube with metal braid and/or coils in the wall of the tube.

With brief reference to FIG. 1, the inflatable balloon 24 includes a proximal waist 34 and a distal waist 35. In some cases, the inner shaft 36 may extend distally to a point at or even distal to the distal waist 35. In some instances, the outer shaft 38 may extend distally to a point at or even slightly distal to the proximal waist 34.

As shown in FIG. 2, the outer shaft 38 includes a reinforcing member 46. In some cases, the reinforcing member 46 may be a braid. The braid may have any number of filars wound in a first helical direction and any number of filars wound in an opposing second helical direction. The braid may serve to reinforce the outer shaft 38 in a way that increases the torqueability of the outer shaft 38, and hence the torqueability of the elongate shaft 20. The braid may be formed of any desired material, and may include metallic filars and/or polymeric filars, for example. In some instances, the reinforcing member 46 may extend distally to a point at or even just beyond the proximal waist 34 (FIG. 1).

In some cases, the reinforcing member 46 may be disposed within (e.g., embedded within) the wall 48 of the outer shaft 38, as shown. In some cases, it is contemplated that the reinforcing member 46 may be secured relative to an interior surface 50 of the outer shaft 38. In some instances, the reinforcing member 46 may be secured relative to an exterior surface 52 of the outer shaft 38. In some cases, the reinforcing member 46 may be disposed between inner and outer layers of the outer shaft 38. In some cases, the outer shaft 38 may be formed by coating the reinforcing member 46 with sufficient polymeric material, such as by spray coating or dip coating, to actually define the outer shaft 38. These are just examples.

As shown in FIG. 2, the inner shaft 36 includes a reinforcing member 47. In some cases, the reinforcing member 47 may be a braid. The braid may have any number of filars wound in a first helical direction and any number of filars wound in an opposing second helical direction. The braid may serve to reinforce the inner shaft 36 in a way that increases the torqueability of the outer shaft 38, and hence the torqueability of the elongate shaft 20. The braid may be formed of any desired material, and may include metallic filars and/or polymeric filars, for example.

In some cases, the reinforcing member 47 may be disposed within (e.g., embedded within) the wall 49 of the inner shaft 36, as shown. In some cases, it is contemplated that the reinforcing member 47 may be secured relative to an interior surface 41 of the inner shaft 36. In some instances, the reinforcing member 46 may be secured relative to an exterior surface 40 of the inner shaft 36. In some cases, the reinforcing member 47 may be disposed between inner and outer layers of the inner shaft 36. In some cases, the inner shaft 36 may be formed by coating the reinforcing member 47 with sufficient polymeric material, such as by spray coating or dip coating, to actually define the inner shaft 36. These are just examples.

As illustrated, the inner shaft 36 includes a reinforcing member 47 and the outer shaft 38 includes a reinforcing member 46. In some cases, the inner shaft 36 may include the reinforcing member 47 while the outer shaft 38 does not include the reinforcing member 46. In some instances, the outer shaft 38 includes the reinforcing member 46 while the inner shaft 36 does not include the reinforcing member 47. In some instances, the reinforcing member 47, if present, may extend up to the proximal waist or through the inflatable balloon 24 to a point at or even distal of the distal waist 35.

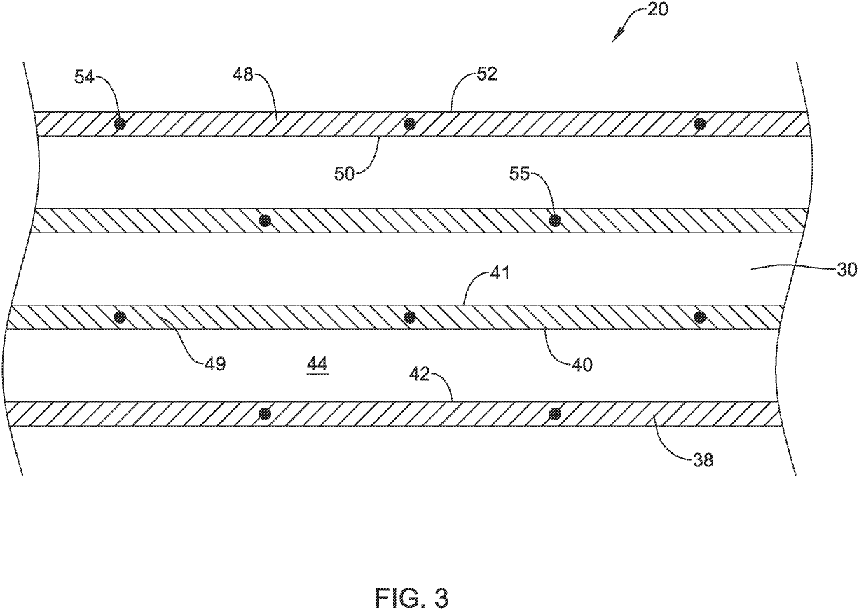

As shown in FIG. 3, the outer shaft 38 includes a reinforcing member 54. In some cases, the reinforcing member 54 may be a coil. The reinforcing member 54 may include a single coil. In some cases, the reinforcing member 54 may include a pair of coils, such as a first coil extending in a first helical direction and a second coil surrounding the first coil and extending in the same or a second helical direction, for example, the coil (or coils) may serve to reinforce the outer shaft 38 in a way that increases the torqueability of the outer shaft 38, and hence the torqueability of the elongate shaft 20. The coil may be formed of any desired material, including various metals and polymers. In some instances, the reinforcing member 54 may extend distally to a point at or even just beyond the proximal waist 34 (FIG. 1).

In some cases, the reinforcing member 54 may be disposed within (e.g., embedded within) the wall 48 of the outer shaft 38, as shown. In some cases, it is contemplated that the reinforcing member 54 may be secured relative to an interior surface 50 of the outer shaft 38. In some instances, the reinforcing member 54 may be secured relative to an exterior surface 52 of the outer shaft 38. In some cases, the reinforcing member 54 may be disposed between inner and outer layers of the outer shaft 38. In some cases, the outer shaft 38 may be formed by coating the reinforcing member 54 with sufficient polymeric material, such as by spray coating or dip coating, to actually define the outer shaft 38. These are just examples.

In some cases, the elongate shaft 20 may include one or more portions that include a reinforcing braid as well as one or more portions that include one or more reinforcing coils. In some instances, one or more portions of the elongate shaft 20 may include both a reinforcing braid as well as one or more reinforcing coils. In some cases, the elongate shaft 20 may include a reinforcing braid surrounded by one or more reinforcing coils and/or one or more reinforcing coils surrounded by a reinforcing braid. In some cases, at least part of the elongate shaft 20 may be formed of or otherwise include a hypotube that has been micro-machined for enhanced flexibility and torque transmission. In some cases, the elongate shaft 20 may include an outer shaft 38 that is formed of multiple layers.

As shown in FIG. 3, the inner shaft 36 includes a reinforcing member 55. In some cases, the reinforcing member 55 may be a coil. The reinforcing member 55 may include a single coil. In some cases, the reinforcing member 54 may include a pair of coils, such as a first coil extending in a first helical direction and a second coil surrounding the first coil and extending in a second helical direction, for example, the coil (or coils) may serve to reinforce the inner shaft 36 in a way that increases the torqueability of the inner shaft 36, and hence the torqueability of the elongate shaft 20. The reinforcing member 55 may extend through the inflatable balloon 24 to a point at or even distal of the distal waist 35. The coil may be formed of any desired material, including various metals and polymers.

In some cases, the reinforcing member 55 may be disposed within (e.g., embedded within) the wall 49 of the inner shaft 36, as shown. In some cases, it is contemplated that the reinforcing member 55 may be secured relative to an interior surface 41 of the inner shaft 36. In some instances, the reinforcing member 55 may be secured relative to an exterior surface 40 of the inner shaft 36. In some cases, the reinforcing member 55 may be disposed between inner and outer layers of the outer shaft 38. In some cases, the inner shaft 36 may be formed by coating the reinforcing member 55 with sufficient polymeric material, such as by spray coating or dip coating, to actually define the inner shaft 36. These are just examples.

As illustrated, the inner shaft 36 includes a reinforcing member 55 and the outer shaft 38 includes a reinforcing member 54. In some cases, the inner shaft 36 may include the reinforcing member 55 while the outer shaft 38 does not include the reinforcing member 54. In some instances, the outer shaft 38 includes the reinforcing member 54 while the inner shaft 36 does not include the reinforcing member 55. In some cases, the inner shaft 36 may include a braided reinforcing member while the outer shaft 38 includes a coil reinforcing member. The inner shaft 36 may include a coil reinforcing member while the outer shaft 38 may include a braided reinforcing member.

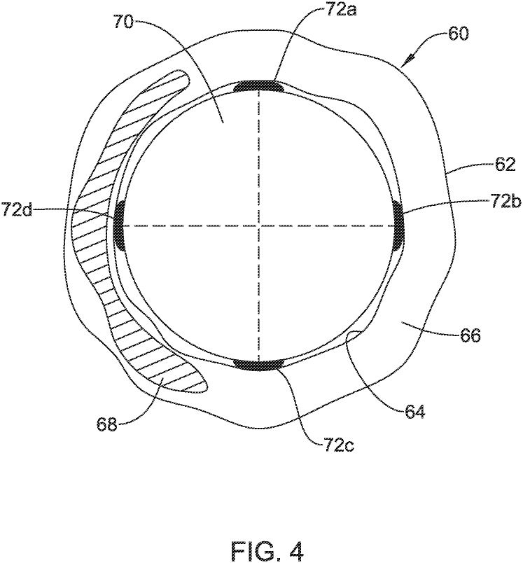

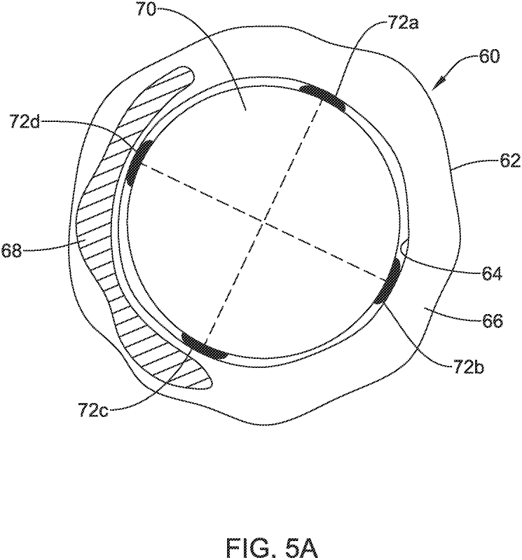

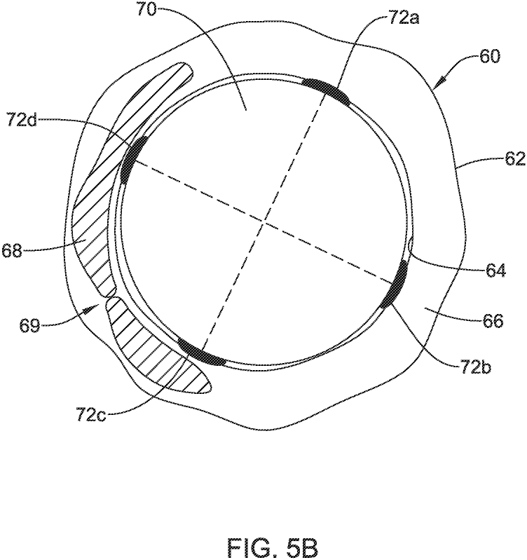

The importance of torqueability, and the ability to reliably rotate the expandable lesion-engaging portion 22, can be seen for example in FIGS. 4, 5A and 5B. FIG. 4 is a schematic cross-sectional view of a vessel 60 that includes an outer surface 62, an inner surface 64 and an intervening vessel wall 66. A vascular asymmetric or eccentric lesion 68, which may for example be a calcified lesion, is formed within the vessel wall 66. In some cases, the lesion 68 reaches the inner surface 64 of the vessel 60. In some instances, the vascular asymmetric or eccentric lesion 68 is held entirely within the vessel wall 66 and thus does not reach the inner surface 64. In describing the lesion 68 as asymmetric or eccentric, what is meant is that the lesion 68 does not extend around an entire circumference of the vessel wall 66. For example, the vascular asymmetric or eccentric lesion 68 may extend around 270 degrees or less of the circumference or 180 degrees or less of the circumference of the vessel wall 66.

An expandable lesion-engaging portion 70 is disposed within the vessel 60, and is shown in its expanded configuration. The expandable lesion-engaging portion 70, which may for example include an inflatable balloon, includes a total of four traction elements 72a, 72b, 72c and 72d. Because there is a total of four traction elements 72a, 72b, 72c, 72d, they are equidistantly spaced about 90 degrees apart around the circumference of the expandable lesion-engaging portion 70. As seen in FIG. 4, only the traction element 72d is in contact with the vascular eccentric lesion 68 applying a single cutting, scoring or compressive force at the location where the traction element 72d contacts the vascular eccentric lesion 68.

Accordingly, an operator may wish to contract the expandable lesion-engaging portion 70 (e.g., deflate the balloon), rotate the expandable lesion-engaging portion 70 to gain a different rotational orientation, and then once again expand the expandable lesion-engaging portion 70. As can be seen in FIG. 5A, the expandable lesion-engaging portion 70 has been rotated sufficiently (in a clockwise direction, as illustrated) such that now the traction element 72d remains in contact with the vascular eccentric lesion 68 but additionally the traction element 72c is also in contact with the vascular eccentric lesion 68. In some instances, the operator may rotate the hub or manifold at the proximal region of the elongate shaft of the catheter less than 90 degrees, less than 75 degrees, less than 60 degrees, less than 90 degrees but greater than 10 degrees, less than 75 degrees but greater than 10 degrees, or less than 60 degrees but greater than 10 degrees, for example. For instance, the operator may rotate the hub or manifold at the proximal region of the elongate shaft of the catheter 10 degrees to 75 degrees, 10 degrees to 60 degrees, 10 degrees to 45 degrees, 20 degrees to 75 degrees, 20 degrees to 60 degrees, 20 degrees to 45 degrees, 30 degrees to 75 degrees, 30 degrees to 60 degrees, or 30 degrees to 45 degrees. Due to the torqueability of the catheter shaft, the particular rotation made at the proximal region of the catheter through rotating the hub or manifold will be communicated to the distal region of the catheter to rotate the inflatable balloon, or other expandable lesion-engaging portion 70, a corresponding amount. The corresponding amount of rotation experienced by the inflatable balloon, or other expandable lesion-engaging portion 70, at the distal end of the catheter shaft may be within plus or minus twenty (20) percent, or within plus or minus ten (10) percent of the rotation imparted on the hub or manifold located at the proximal end of the catheter shaft. Having two or more, or a plurality of traction elements 72 simultaneously in contact with the vascular eccentric lesion 68 increases the chances of being able to stretch or crack the vascular eccentric lesion 68, or a portion thereof, when expanding the expandable lesion-engaging portion 70.

In some cases, as shown, for example, in FIG. 5A, having at least two traction elements in contact with the vascular eccentric lesion 68 helps to apply multiple cutting, scoring or compressive forces to the vascular eccentric lesion 68 (i.e., a cutting, scoring or compressive force by each traction element in contact with the vascular eccentric lesion 68) but also to isolate the balloon circumferential dilation forces stretching the vascular eccentric lesion 68 between two traction elements contacting the vascular eccentric lesion 68 and cause the vascular eccentric lesion 68 to crack, such as at a location circumferentially between two traction elements (e.g., cutting blades, wires or atherotomes) contacting the eccentric lesion 68. For example, FIG. 5B shows the balloon further inflated, which increases the circumferential distance between the two traction elements 72c and 72d engaging the eccentric lesion 68, and thus circumferentially stretching the vessel wall 66, forming a crack 69 in the vascular eccentric lesion 68 as the two portions of the eccentric lesion 68 contacting the traction elements 72c and 72d are circumferentially spread apart or stretched.

Accordingly, an operator may wish to contract the expandable lesion-engaging portion 70, rotate the expandable lesion-engaging portion 70 to gain a different rotational orientation, and then once again expand the expandable lesion-engaging portion 70. This is particularly useful in cases in which the vascular eccentric lesion 68 extends less than 180 degrees about the circumference of the vessel 60. It will be appreciated that if the vascular eccentric lesion 68 extends more than 180 degrees about the circumference of the vessel 60, the chances that at least two traction elements engage the vascular eccentric lesion 68 are increased. Being able to controllably rotate the expandable lesion-engaging portion 70 into multiple rotational orientations, with expanding the expandable lesion-engaging portion 70 at each rotational orientation, substantially increases the chances of successfully causing the lesion 68 to crack as a result of the vessel wall 66 being stretched circumferentially between the two traction elements contacting the vascular eccentric lesion 68.

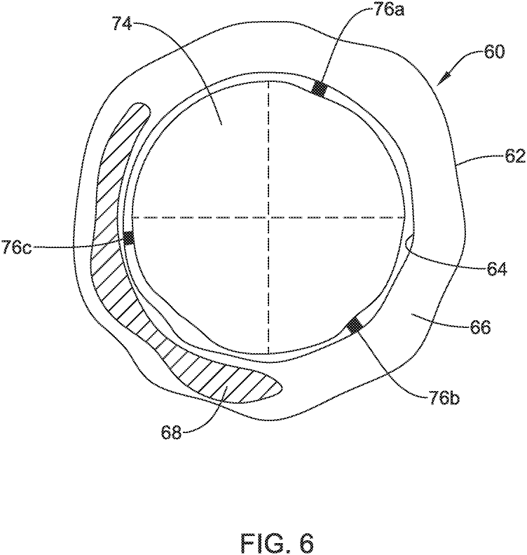

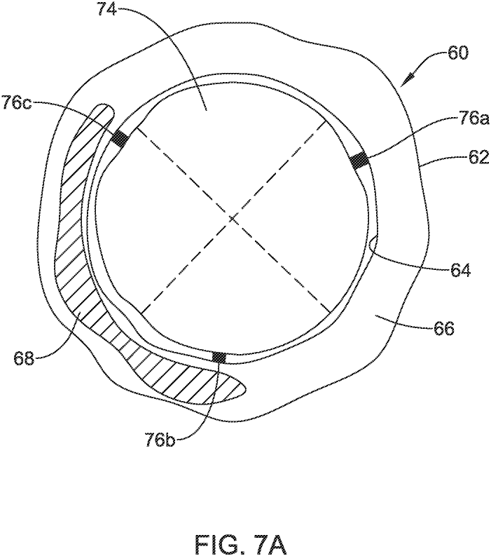

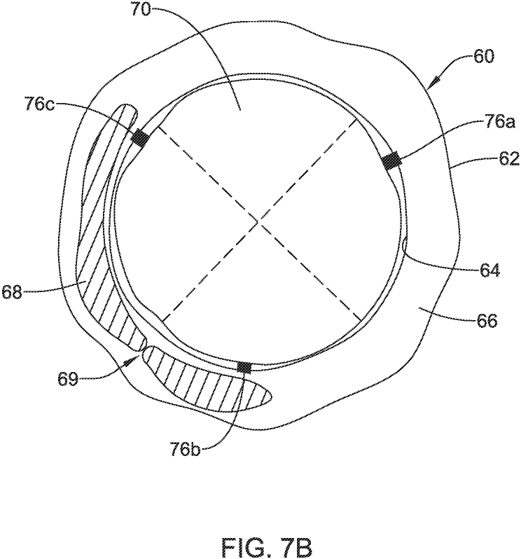

Another illustrative example of the ability to reliably rotate the expandable lesion-engaging portion 22 is illustrated in FIGS. 4, 5A and 5B. FIGS. 6, 7A and 7B provide an example of an illustrative expandable lesion-engaging portion 74 having a total of three traction elements 76a, 76b, 76c disposed within the vessel 60, adjacent the lesion 68. The three traction elements 76a, 76b, 76c are equidistantly spaced apart. As seen in FIG. 6, only a single traction element 76c is in contact with the vascular eccentric lesion 68 applying a single cutting, scoring or compressive force at the location where the traction element 76c contacts the vascular eccentric lesion 68.

Accordingly, an operator may wish to contract the expandable lesion-engaging portion 74 (e.g., deflate the balloon), rotate the expandable lesion-engaging portion 74 to gain a different rotational orientation, and then once again expand the expandable lesion-engaging portion 74. As can be seen in FIG. 7A, the expandable lesion-engaging portion 74 has been rotated sufficiently (in a clockwise direction, as illustrated) such that now the traction element 76c remains in contact with the lesion 68 but additionally the traction element 76b is now in contact with the vascular eccentric lesion 68. In some instances, the operator may rotate the hub or manifold at the proximal region of the elongate shaft of the catheter less than 120 degrees, less than 100 degrees, 90 degrees, less than 75 degrees, less than 60 degrees, less than 120 degrees but greater than 10 degrees, less than 100 degrees but greater than 10 degrees, less than 90 degrees but greater than 10 degrees, less than 75 degrees but greater than 10 degrees, or less than 60 degrees but greater than 10 degrees, for example. For instance, the operator may rotate the hub or manifold at the proximal region of the elongate shaft of the catheter 10 degrees to 100 degrees, 10 degrees to 90 degrees, 10 degrees to 75 degrees, 10 degrees to 60 degrees, 10 degrees to 45 degrees, 20 degrees to 100 degrees, 20 degrees to 90 degrees, 20 degrees to 75 degrees, 20 degrees to 60 degrees, 20 degrees to 45 degrees, 30 degrees to 100 degrees, 30 degrees to 90 degrees, 30 degrees to 75 degrees, 30 degrees to 60 degrees, or 30 degrees to 45 degrees. Due to the torqueability of the catheter shaft, the particular rotation made at the proximal region of the catheter through rotating the hub or manifold will be communicated to the distal region of the catheter to rotate the inflatable balloon, or other expandable lesion-engaging portion 74, a corresponding amount. The corresponding amount of rotation experienced by the inflatable balloon, or other expandable lesion-engaging portion 74, at the distal end of the catheter shaft may be within plus or minus twenty (20) percent, or within plus or minus ten (10) percent of the rotation imparted on the hub or manifold located at the proximal end of the catheter shaft. Having two or more, or a plurality of traction elements 76 simultaneously in contact with the vascular eccentric lesion 68 increases the chances of being able to stretch or crack the vascular eccentric lesion 68, or a portion thereof, when expanding the expandable lesion-engaging portion 74.

In some cases, as shown, for example, in FIG. 7A, having at least two traction elements in contact with the vascular eccentric lesion 68 helps apply multiple cutting, scoring or compressive forces to the vascular eccentric lesion 68 (i.e., cutting, scoring or compressive force by each traction element in contact with the vascular eccentric lesion 68) but also to isolate the balloon circumferential dilation forces stretching the vascular eccentric lesion 68 between two traction elements contacting the vascular eccentric lesion 68 and cause the vascular eccentric lesion 68 to crack, such as at a location circumferentially between two traction elements (e.g., cutting blades, wires or atherotomes) contacting the eccentric lesion 68. For example, FIG. 7B shows the balloon further inflated, which increases the circumferential distance between the two traction elements 76b and 76c engaging the eccentric lesion 68, and thus circumferentially stretching the vessel wall 66, forming a crack 69 in the vascular eccentric lesion 68 as the two portions of the eccentric lesion 68 contacting the traction elements 76b and 76c are circumferentially spread apart or stretched.

Accordingly, an operator may wish to contract the expandable lesion-engaging portion 74, rotate the expandable lesion-engaging portion 74 to gain a different rotational orientation, and then once again expand the expandable lesion-engaging portion 74. This is particularly useful in cases in which the vascular eccentric lesion 68 extends less than 180 degrees about the circumference of the vessel 60. It will be appreciated that if the vascular eccentric lesion 68 extends more than 180 degrees about the circumference of the vessel 60, the chances that at least two traction elements engage the vascular eccentric lesion 68 are increased. Being able to controllably rotate the expandable lesion-engaging portion 74 into multiple rotational orientations, with expanding the expandable lesion-engaging portion 74 at each rotational orientation, substantially increases the chances of successfully causing the vascular eccentric lesion 68 to crack as a result of the vessel wall 66 being stretched circumferentially between two traction elements contacting the vascular eccentric lesion 68.

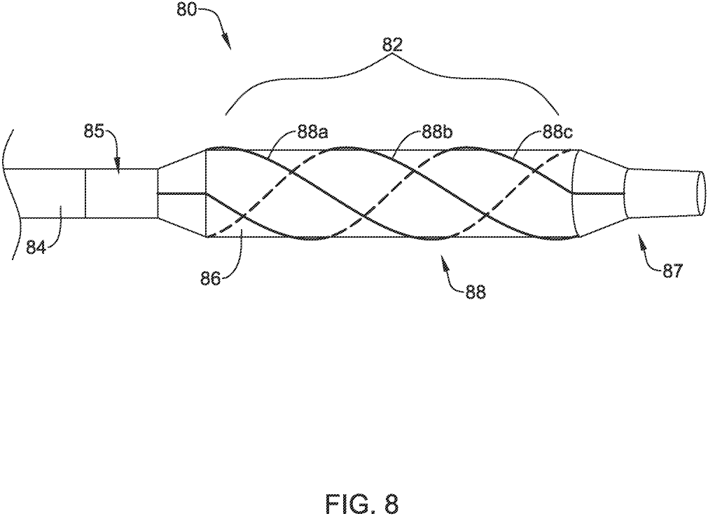

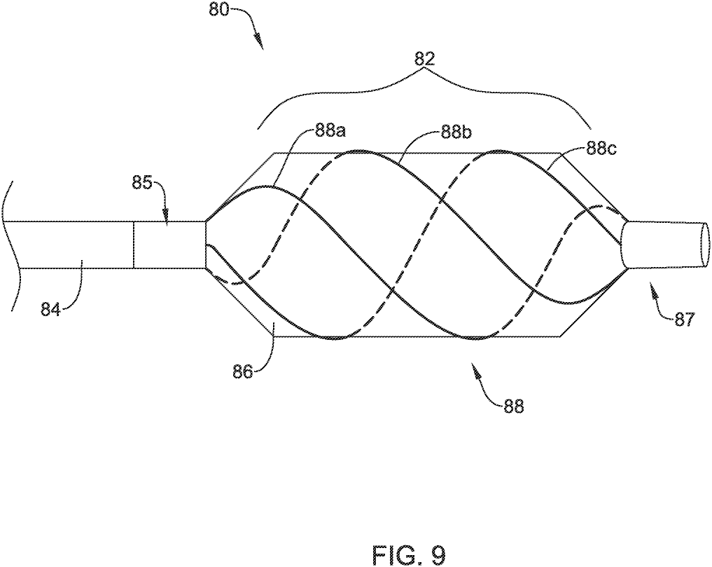

FIG. 8 is a schematic side view of a portion of an illustrative catheter 80 usable for disrupting vascular lesions, shown in a deflated configuration while FIG. 9 is a schematic side view of a portion of the illustrative catheter 80 shown in an inflated configuration. The catheter 80 includes an expandable lesion-engaging portion 82 that is secured relative to an elongate shaft 84. In some cases, as illustrated, the expandable lesion-engaging portion 82 includes an inflatable balloon 86 and several helical elements 88 (individually labeled as 88a, 88b and 88c) that are disposed about the inflatable balloon 86. In some instances, the helical elements 88 extend from at or near a proximal waist 85 to a position at or near a distal waist 87 of the inflatable balloon 86. In comparing FIG. 8 (deflated) and FIG. 9 (inflated), it can be seen that the inflatable balloon 86 radially expands when inflated, and the inflatable balloon 86 causes the helical elements 88 to be expanded radially outwardly (and possibly shorten slightly axially) when the expandable lesion-engaging portion 82 moves into its expanded configuration as shown in FIG. 9.

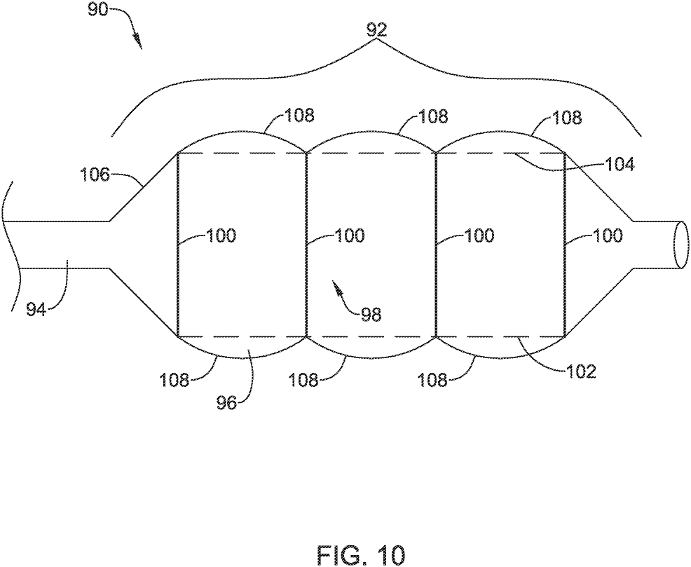

FIG. 10 is a schematic side view of a portion of an illustrative catheter 90 usable for treating vascular lesions, shown in an inflated configuration. The catheter 90 includes an expandable lesion-engaging portion 92 that is secured relative to an elongate shaft 94. In some cases, as illustrated, the expandable lesion-engaging portion 92 includes an inflatable balloon 96 and a cage-like structure 98 secured about the inflatable balloon 96. The cage-like structure 98 includes a plurality of annular rungs 100 that extend circumferentially about the inflatable balloon 96 and that are coupled together via a first framework 102 and a second framework 104. When the inflatable balloon 96 inflates, as shown, an outer surface 106 of the inflatable balloon 96 is constrained by the cage-like structure 98 and forms a plurality of pillowed portions 108. It will be appreciated that the pillowed portions 108 function as traction elements.

FIG. 11 is a flow diagram showing an illustrative method 110 of treating a lesion within a blood vessel. The method 110 includes advancing a catheter (such as the catheter 10 shown in FIG. 1) to a treatment site proximate a lesion, the catheter including a proximal region and a distal region, the distal region including a plurality of traction elements and an inflatable balloon adapted to urge the plurality of traction elements radially outwardly when the inflatable balloon is inflated, as indicated at block 112. In some cases, urging the plurality of traction elements radially outwardly can cause at least a portion of the lesion to stretch or crack. In some cases, for a given rotation of the proximal region of the catheter, the distal region of the catheter may rotate in the same direction a rotational distance that is within twenty percent of the given rotation. In some cases, for a given rotation of the proximal region of the catheter, the distal region of the catheter rotates in the same direction a rotational distance that is within ten percent of the given rotation. In some cases, for a given rotation of the proximal region of the catheter, the distal region of the catheter rotates in the same direction a rotational distance that is within five percent of the given rotation.

The inflatable balloon is inflated to urge the plurality of traction elements radially outwardly into contact with the lesion, as indicated at block 114. The inflatable balloon is deflated, as indicated at block 116. The proximal region of the catheter is rotated in order to achieve a corresponding rotation of the distal region of the catheter relative to the treatment site, as indicated at block 118. The inflatable balloon is re-inflated again to urge the plurality of traction elements radially outwardly into contact with the lesion, as indicated at block 120.

It will be appreciated that for relatively small lesions that extend less than 180 degrees around the circumference of a blood vessel, it can be hit or miss as to whether two or more traction elements happen to engage the lesion when the inflatable balloon is inflated. Accordingly, being able to deflate the inflatable balloon, rotate the inflatable balloon (and corresponding traction elements) to another rotational orientation, followed by inflating the inflatable balloon in order to once again urge the traction elements outwardly and into contact with the lesion increases the changes of being able to stretch the vessel wall and thus crack the lesion, particularly without visualization of the inflatable balloon and corresponding traction elements relative to the lesion.

In some cases, the plurality of traction elements may be oriented in an axial direction and are circumferentially spaced on an outer surface of the inflatable balloon. The plurality of traction elements may together form a wire cage disposed about the inflatable balloon. In some instances, the plurality of traction elements includes an outer surface of the balloon. The catheter may include an elongate shaft as well as one or more torque-transmission elements (such as braids, coils, hypotubes and the like) extending within the elongate shaft.

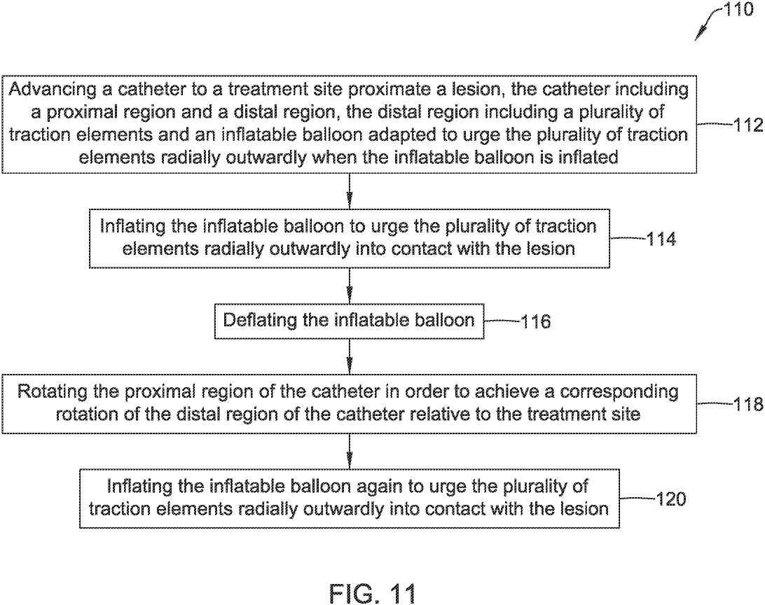

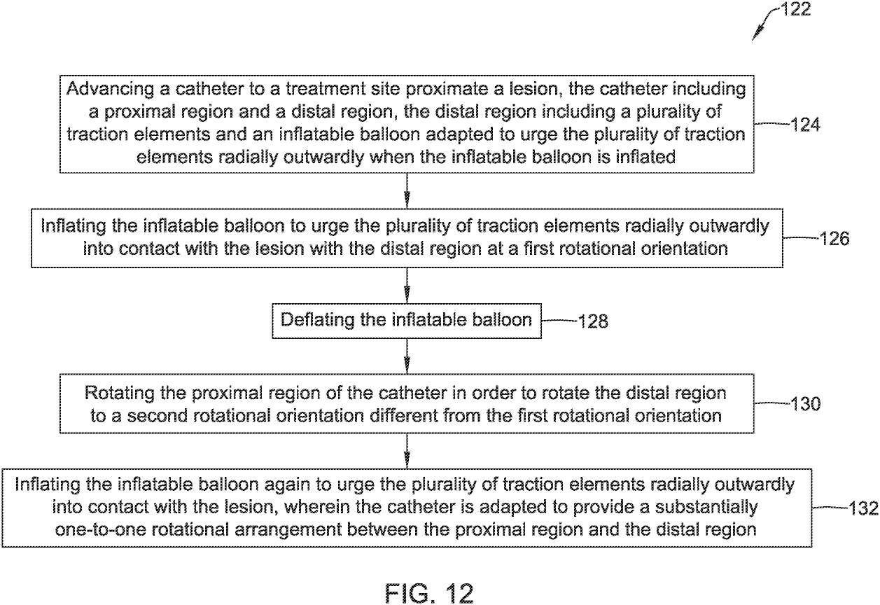

FIG. 12 is a flow diagram showing an illustrative method 122 of treating a lesion within a blood vessel. The method 122 includes advancing a catheter (such as the catheter 10 shown in FIG. 1) to a treatment site proximate a lesion, the catheter including a proximal region and a distal region, the distal region including a plurality of traction elements and an inflatable balloon adapted to urge the plurality of traction elements radially outwardly when the inflatable balloon is inflated, as indicated at block 124. In some cases, urging the plurality of traction elements radially outwardly can cause at least a portion of the lesion to stretch or crack. In some cases, for a given rotation of the proximal region of the catheter, the distal region of the catheter may rotate in the same direction a rotational distance that is within twenty percent of the given rotation. In some cases, for a given rotation of the proximal region of the catheter, the distal region of the catheter rotates in the same direction a rotational distance that is within ten percent of the given rotation. In some cases, for a given rotation of the proximal region of the catheter, the distal region of the catheter rotates in the same direction a rotational distance that is within five percent of the given rotation.

The inflatable balloon is inflated to urge the plurality of traction elements radially outwardly into contact with the lesion with the distal region at a first rotational orientation, as indicated at block 126. The inflatable balloon is deflated, as indicated at block 128. The proximal region of the catheter is rotated in order to rotate the distal region to a second rotational orientation different from the first rotational orientation, as indicated at block 130. The inflatable balloon is re-inflated to again urge the plurality of traction elements radially outwardly into contact with the lesion, wherein the catheter is adapted to provide a substantially one-to-one rotational arrangement between the proximal region and the distal region, as indicated at block 132.

In some cases, the plurality of traction elements may be oriented in an axial direction and are circumferentially spaced on an outer surface of the inflatable balloon. The plurality of traction elements may together form a wire cage disposed about the inflatable balloon. In some instances, the plurality of traction elements includes an outer surface of the balloon. The catheter may include an elongate shaft as well as one or more torque-transmission elements (such as braids, coils, hypotubes and the like) extending within the elongate shaft.

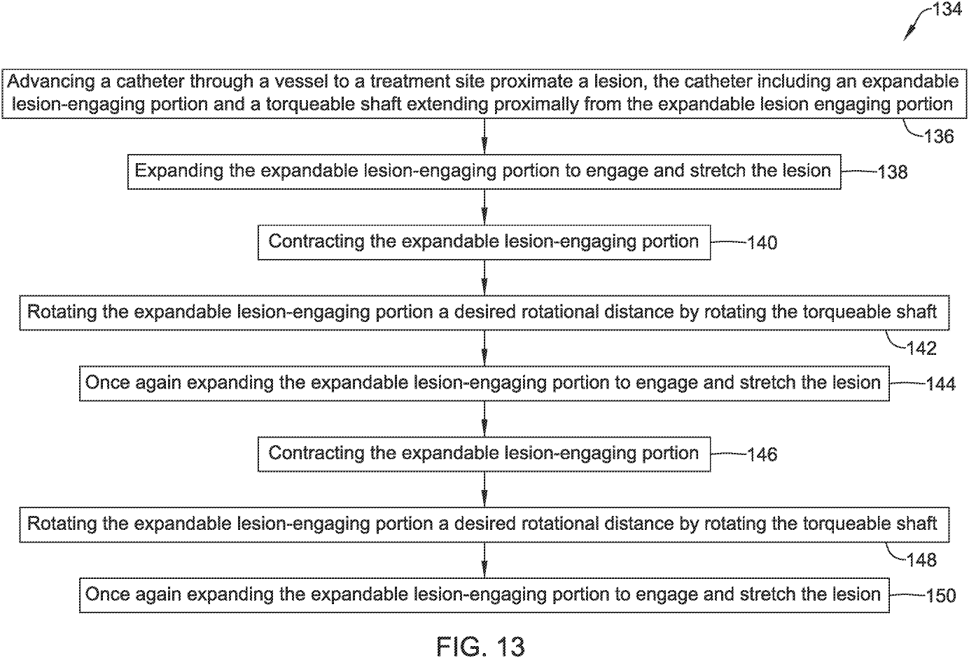

FIG. 13 is a flow diagram showing an illustrative method 134 of treating a lesion. The method 134 includes advancing a catheter (such as the catheter 10 shown in FIG. 1) through a vessel to a treatment site proximate a lesion, the catheter including an expandable lesion-engaging portion and a torqueable shaft extending proximally from the expandable lesion-engaging portion, as indicated at block 136. The expandable lesion-engaging portion is expanded to engage and stretch the lesion, as indicated at block 138. The expandable lesion-engaging portion is contracted, as indicated at block 140. The expandable lesion-engaging portion is rotated a desired rotational distance by rotating the torqueable shaft, as indicated at block 142. The expandable lesion-engaging portion is expanded again to engage and stretch the lesion, as indicated at block 144.

In some cases, the method 134 may further include contracting the expandable lesion-engaging portion, as indicated at block 146. The method 134 may further include rotating the expandable lesion-engaging portion a desired rotational distance by rotating the torqueable shaft, as indicated at block 148. The method 134 may further include once again expanding the expandable lesion-engaging portion to engage and stretch the lesion, as indicated at block 150.

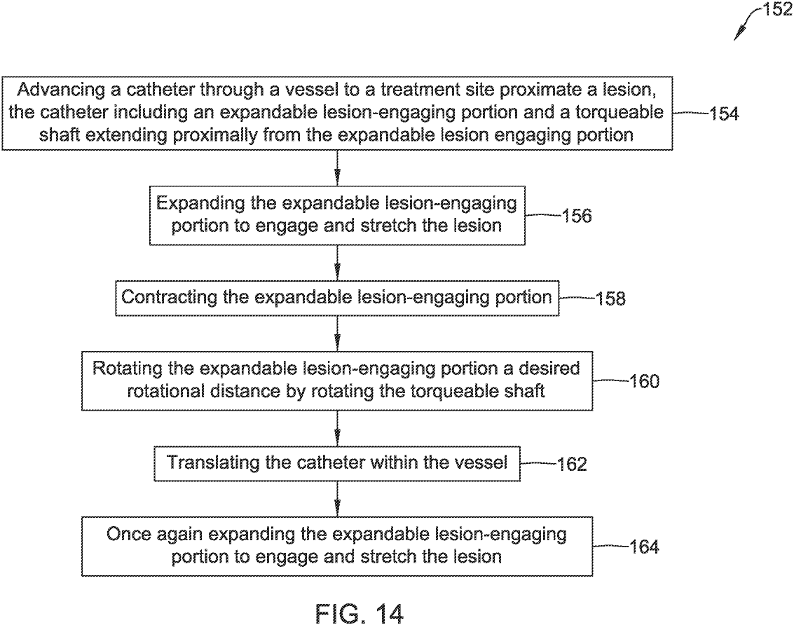

FIG. 14 is a flow diagram showing an illustrative method 152 of treating a lesion. The method 152 includes advancing a catheter (such as the catheter 10 shown in FIG. 1) through a vessel to a treatment site proximate a lesion, the catheter including an expandable lesion-engaging portion and a torqueable shaft extending proximally from the expandable lesion-engaging portion, as indicated at block 154. The expandable lesion-engaging portion is expanded to engage and stretch the lesion, as indicated at block 156. In some cases, the expandable lesion-engaging portion includes a plurality of traction elements and an inflatable balloon adapted to urge the plurality of traction elements radially outwardly when the inflatable balloon is inflated.

The expandable lesion-engaging portion is contracted, as indicated at block 158. The expandable lesion-engaging portion is rotated a desired rotational distance by rotating the torqueable shaft, as indicated at block 160. In some instances, rotating the distal region a desired rotation distance includes rotating the torqeueable shaft a corresponding rotational distance. In some cases, the catheter may also be translated longitudinally within the vessel, as indicated at block 162. The expandable lesion-engaging portion is expanded again to engage and stretch the lesion, as indicated at block 164.

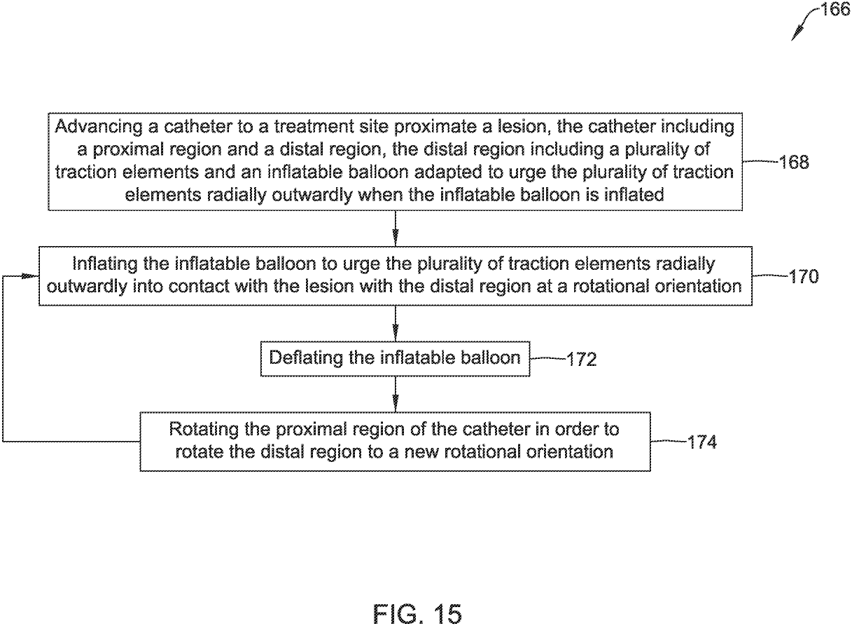

FIG. 15 is a flow diagram showing an illustrative method 166 oftreating a lesion within a blood vessel. The method 166 includes advancing a catheter (such as the catheter 10 shown in FIG. 1) to a treatment site proximate a lesion, the catheter including a proximal region and a distal region, the distal region including a plurality of traction elements and an inflatable balloon adapted to urge the plurality of traction elements radially outwardly when the inflatable balloon is inflated, as indicated at block 168. In some cases, urging the plurality of traction elements radially outwardly can cause at least a portion of the lesion to stretch or crack. In some cases, for a given rotation of the proximal region of the catheter, the distal region of the catheter may rotate in the same direction a rotational distance that is within twenty percent of the given rotation. In some cases, for a given rotation of the proximal region of the catheter, the distal region of the catheter rotates in the same direction a rotational distance that is within ten percent of the given rotation. In some cases, for a given rotation of the proximal region of the catheter, the distal region of the catheter rotates in the same direction a rotational distance that is within five percent of the given rotation.

The inflatable balloon is inflated in order to urge the plurality of traction elements radially outwardly into contact with the lesion with the distal region of the catheter at a rotational orientation, as indicated at block 170. The inflatable balloon is deflated, as indicated at block 172. Next, the proximal region of the catheter is rotated in order to rotate the distal region of the catheter to a new rotational orientation, as indicated at block 174. The steps 170, 172 and 174 can be repeated multiple times. It will be appreciated that for relatively small lesions that extend less than 180 degrees around the circumference of a blood vessel, it can be hit or miss as to whether two or more traction elements happen to engage the lesion when the expandable lesion-engaging portion is expanded. Accordingly, being able to contract the expandable lesion-engaging portion, rotate the expandable lesion-engaging portion to another rotational orientation, followed by expanding the expandable lesion-engaging portion into contact with the lesion increases the chances of being able to stretch the vessel wall and thus crack the lesion, particularly without visualization of the expandable lesion-engaging portion relative to the lesion.

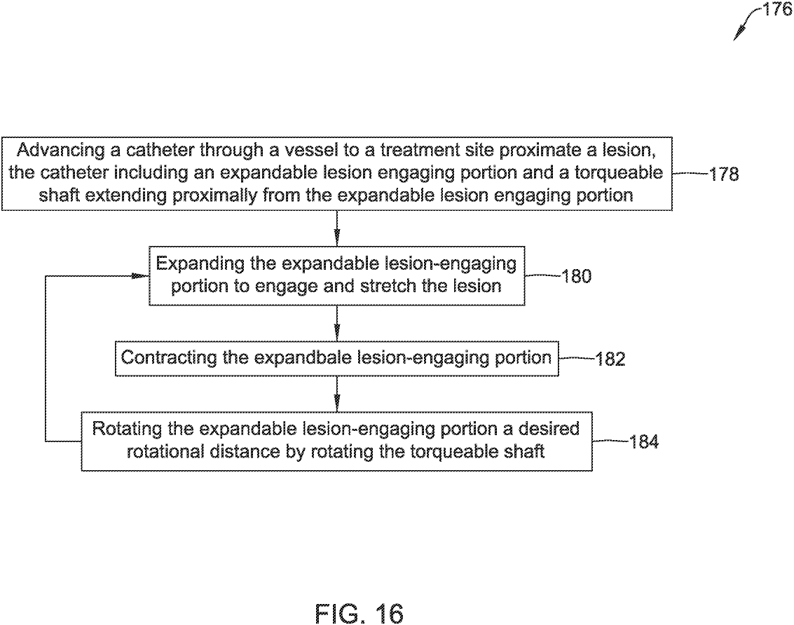

FIG. 16 is a flow diagram showing an illustrative method 176 of treating a lesion. The method 176 includes advancing a catheter (such as the catheter 10 shown in FIG. 1) through a vessel to a treatment site proximate a lesion, the catheter including an expandable lesion-engaging portion and a torqueable shaft extending proximally from the expandable lesion-engaging portion, as indicated at block 178. The expandable lesion-engaging portion is expanded to engage and stretch the lesion, as indicated at block 180. In some cases, the expandable lesion-engaging portion includes a plurality of traction elements and an inflatable balloon adapted to urge the plurality of traction elements radially outwardly when the inflatable balloon is inflated.

The expandable lesion-engaging portion is contracted, as indicated at block 182. The expandable lesion-engaging portion is rotated a desired rotational distance by rotating the torqueable shaft, as indicated at block 184. The steps 180, 182 and 184 can be repeated multiple times. It will be appreciated that for relatively small lesions that extend less than 180 degrees around the circumference of a blood vessel, it can be hit or miss as to whether two or more traction elements happen to engage the lesion when the expandable lesion-engaging portion is expanded. Accordingly, being able to contract the expandable lesion-engaging portion, rotate the expandable lesion-engaging portion to another rotational orientation, followed by expanding the expandable lesion-engaging portion into contact with the lesion increases the chances of being able to stretch the vessel wall and thus crack the lesion, particularly without visualization of the expandable lesion-engaging portion relative to the lesion.

As discussed, the catheter 10 has an elongate shaft 20 that is torqueable, meaning that a given rotation of the proximal region 12 causes a corresponding similar rotation of the distal region 14, within perhaps 20 percent, or perhaps 10 percent or even less. In some instances, the elongate shaft 20 may not be torqueable, and may instead be adapted to more easily pass through a tortuous vasculature by being more flexible and perhaps less torqueable. In some cases, a catheter may include a mechanism that accommodates a more flexible and less torqueable elongate shaft 20. In some cases, a catheter may include a mechanism that allows for independent rotational movement between the proximal region 12 and the distal region 14. The mechanism may be adapted to convert axial movement of the elongate shaft 20 into rotation of the distal region 14, which may include rotating the inflatable balloon 24 and/or rotating a structure disposed relative to the inflatable balloon 24 that includes traction elements. FIGS. 8 and 9, for example, provide an example of a number of helical elements 88 that may be considered as forming a cage that is disposable over the inflatable balloon 86. In some cases, it is contemplated that the cage may be rotatable relative to the inflatable balloon 86. FIG. 10 provides an example of a cage-like structure 98 that is disposable over the inflatable balloon 96. In some cases, it is contemplated that the cage-like structure 98 may be rotatable relative to the inflatable balloon 96.

In some instances, it is contemplated that a catheter may include a rotation mechanism that is pneumatically actuated such that each time the inflatable balloon 24 is inflated and/or deflated, the rotation mechanism causes the inflatable balloon 24 and/or traction elements or perhaps a cage disposed relative to the inflatable balloon 24, to rotate relative to the elongate shaft 20 of the catheter proximal of the inflatable balloon 24. Such a rotation mechanism may be disposed between the elongate shaft 20 and the inflatable balloon 24. As an example, such a rotation mechanism could be disposed just proximal of the proximal waist 34 of the inflatable balloon 24. In some instances, an inflation lumen extending within the elongate shaft 20 may extend into and/or through the mechanism, and may be fluidly coupled with the mechanism. This is just an example. In some instances, a catheter may include a rotaion mechanism that is mechanically actuated in order to rotate the inflatable balloon 24 and/or traction elements relative to the elongate shaft 20 of the catheter.

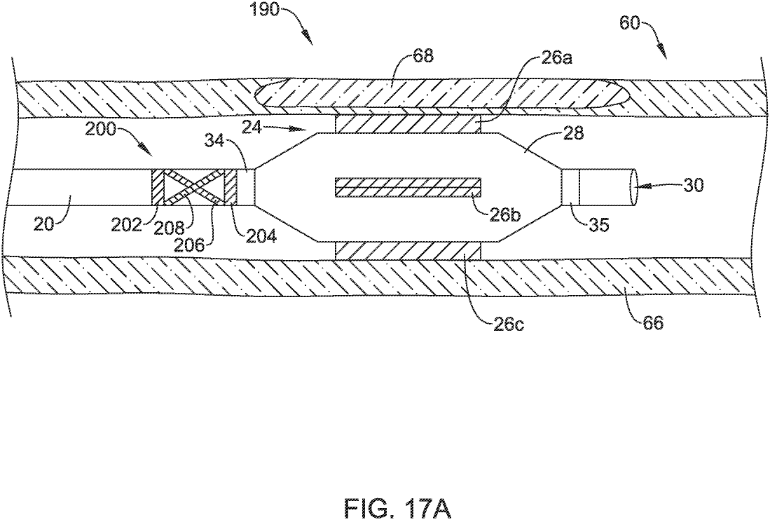

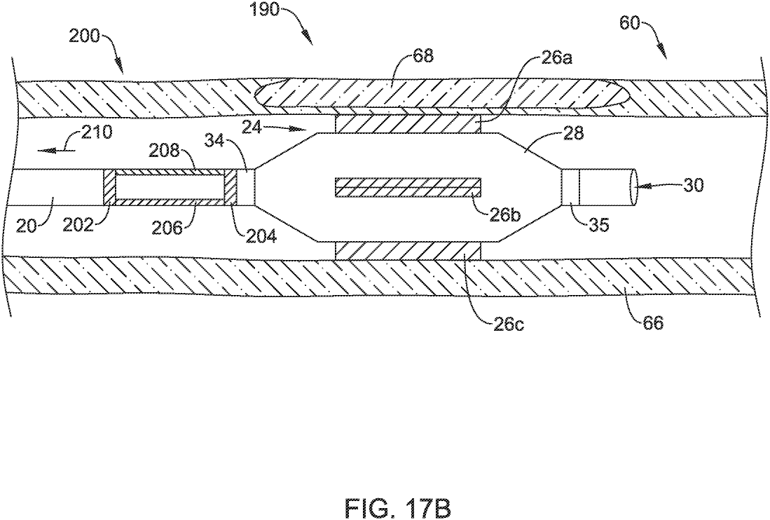

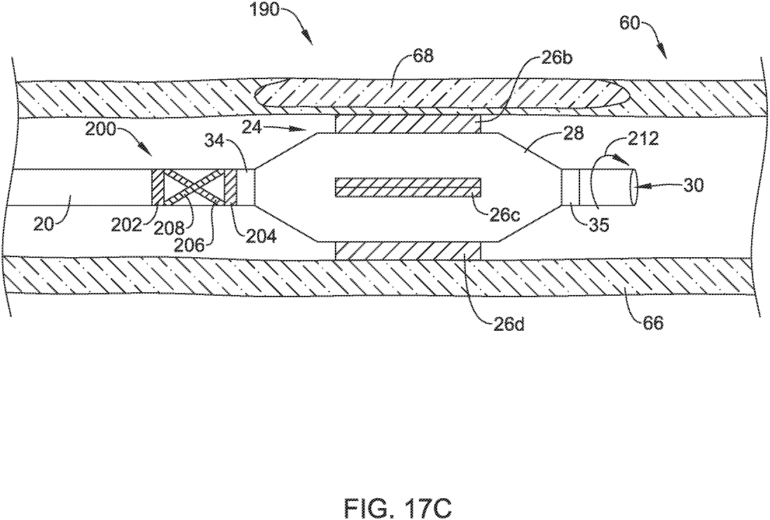

FIGS. 17A through 17C are schematic side views of an illustrative catheter 190 shown disposed within a blood vessel 60 that is defined by a vessel wall 66. A vascular asymmetric or eccentric lesion 68, which may for example be a calcified lesion, is formed within the vessel wall 66. In some cases, the lesion 68 may only extend partially circumferentially around the vessel 60. In some cases, the lesion 68 may include a single lesion 68. While not shown, in some cases there may be two or more distinct lesions 68 formed within the vessel wall 66.

The catheter 190 includes the inflatable balloon 24, as well as a plurality of traction elements 26 that are secured relative to the outer surface 28 of the inflatable balloon 24. Urging the traction elements 26 in an outward direction, such as by inflating the inflatable balloon 24, may cause one or more of the traction elements 26 to engage and stretch the lesion 68. In some cases, one or more of the traction elements 26 may crack the lesion 68. The inflatable balloon 24 includes a proximal waist 34 and a distal waist 35. The catheter 190 includes a guidewire lumen 30 that extends through the inflatable balloon 24. The guidewire lumen 30 may extend proximally to a proximal guidewire port that is disposed within the hub located in the proximal region 12 (FIG. 1), or the guidewire lumen 30 may extend proximally to a proximal guidewire port that is located just proximal of the proximal waist 34.

The catheter 190 includes a mechanically-actuated rotation mechanism 200 that is disposed just proximal of the proximal waist 34. In some cases, the rotation mechanism 200 may be adapted to permit relative rotation between the elongate shaft 20 and the inflatable balloon 24. In some instances, for example, the elongate shaft 20 may include an outer tubular member that terminates distally at the rotation mechanism 200 while an inner tubular member forming an inflation lumen and/or a guidewire lumen extends through the rotation mechanism 200 and to the inflatable balloon 24.

In some instances, the rotation mechanism 200 includes a first component 202 (e.g., a first collar) that is operably coupled with or otherwise fixed relative to the elongate shaft 20 and a second component 204 (e.g., a second collar) that is operably coupled with or otherwise fixed relative to the proximal waist 34 of the inflatable balloon 24. In some instances, he first component 202 may be a ring structure crimped, bonded, adhered or otherwise secured to the elongate shaft 20. The second component 204 may be a ring structure crimped, bonded, adhered or otherwise secured to the proximal waist 34 of the inflatable balloon 24. A plurality of struts may extend between the first component 202 and the second component 204. For example, a first strut 206 and a second strut 208 may each extend between the first component 202 and the second component 204. The rotation mechanism 200 may be formed of any suitable material.

In some cases, the rotation mechanism 200 may be biased into a position, as shown in FIG. 17A, in which the first strut 206 and the second strut 208 extend at an acute angle to the longitudinal axis of the catheter shaft 24. For example, the first strut 206 and/or the second strut 208 may extend in a helical direction around the longitudinal axis, and in some instances the first strut 206 and the second strut 208 may cross each other. It will be appreciated that the inflatable balloon 24 may be considered as having a particular rotational position corresponding to the mechanism 200 being in its biased position, as indicated by the particular arrangement of the traction elements 26a, 26b and 26c visible in FIG. 17A.

In FIG. 17B, a proximally directed axial force has been applied to the elongate shaft 20 while the balloon 24 is inflated within the blood vessel 60, pulling the elongate shaft 20 in a proximal direction indicated by an arrow 210 relative to the balloon 24. The proximal movement of the elongate shaft 20 relative to the balloon 24 may cause the first component 202 to move axially further away from the second component 204 such that the distance between the first component 202 and the second component 204 increases. As the first component 202 moves axially away from the second component 204, the first strut 206 and the second strut 208 may straighten relative to the longitudinal axis of the elongate shaft 20 (e.g., the acute angle between the first strut 206 and/or the second strut 208 and the longitudinal axis may decrease. In FIG. 17B, it can be seen that the rotation mechanism 200 may be elongated, such that the first strut 206 and the second member 208 are now parallel or at least substantially parallel with each other, and parallel to the longitudinal axis of the elongate shaft 20. As a result, tension may be created within the rotation mechanism 200 (such as tension in the first strut 206 and second strut 208) because the inflatable balloon 24 is not able to rotate with the traction elements 26a, 26b and 26c and the inflatable balloon 24 engaged with the vessel wall 66. Thus, potential energy may be stored in the rotation mechanism 200, that once released, tends to drive the rotation mechanism 200 to regain its biased position (as shown in FIG. 17A). In particular, the traction elements 26a, 26b and 26c are engaged with the vessel wall 66 and thus the inflatable balloon 24 is not able to rotate while the inflatable balloon 24 is inflated.

Deflating or at least partially deflating the inflatable balloon 24 causes the traction elements 26a, 26b and 26c to disengage with the vessel wall 66. This allows the inflatable balloon 24, and/or any features disposed on the inflatable balloon 24, to rotate in a direction indicated by an arrow 212, as seen in FIG. 17C. Thus, the potential energy stored in the rotation mechanism 200 may be released to case the inflation balloon 24 and associated traction elements 26 to rotate when the inflatable balloon 24 is deflated. Rotation of the inflatable balloon 24 can be seen by virtue of the traction element 26a no longer being visible, while traction elements 26b, 26c and now 26d are visible, and rotated from their previous positions. This shows that the inflatable balloon 24 has rotated as a result of the rotation mechanism 200 returning to its biased position.

The process may be repeated as many times as is desired, with inflating the inflatable balloon 24 in a particular rotational orientation, followed by pulling proximally (or pushing distally in some configurations) on the elongate shaft 20 in order to move the rotation mechanism 200 away from its biased, equilibrium position. The inflatable balloon 24 may then be at least partially deflated in order to allow the inflatable balloon 24 to rotate as the rotation mechanism 200 returns to its biased, equilibrium position.

It is noted that in other instances, the struts 206/208 may extend generally or substantially parallel to the longitudinal axis of the elongate shaft 20 in the biased, equilibrium position, and then the struts 206/208 may be rotated or deformed into a helical orientation when the elongate shaft 20 is manipulated to transition the rotation mechanism 200 away from its biased, equilibrium position. Subsequent deflation the inflatable balloon 24 may allow the rotation mechanism 200 to revert back to its biased, equilibrium position in which the struts 206/208 are generally or substantially parallel to the longitudinal axis of the elongate shaft 20.

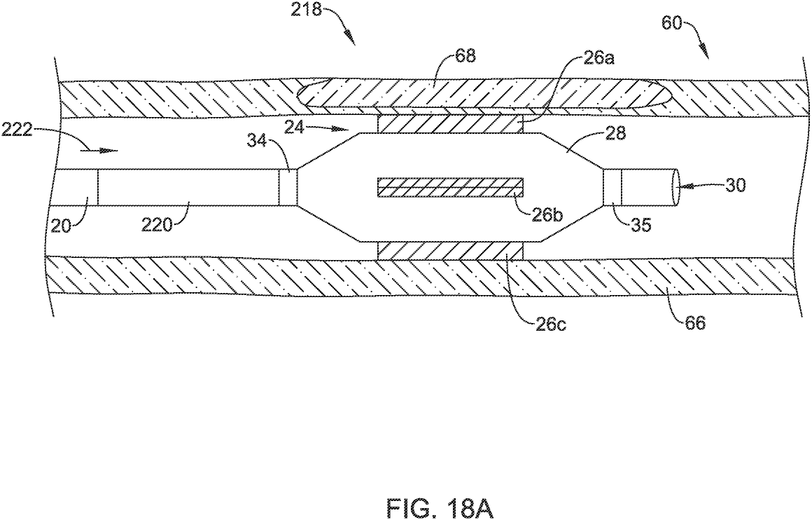

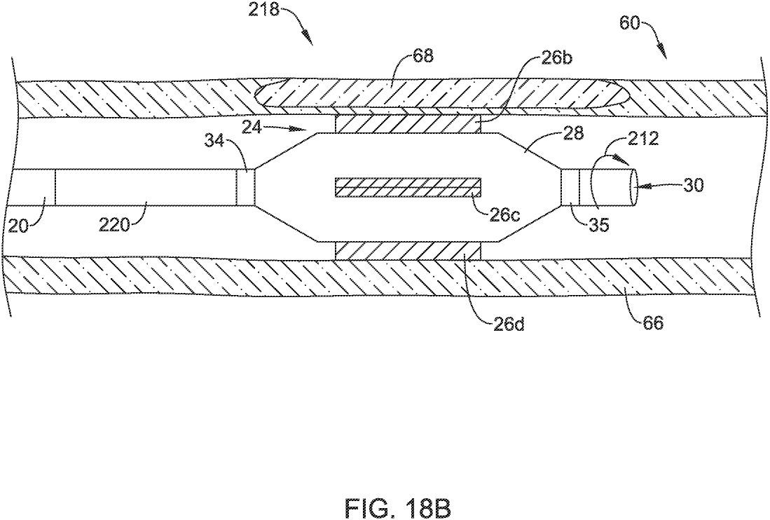

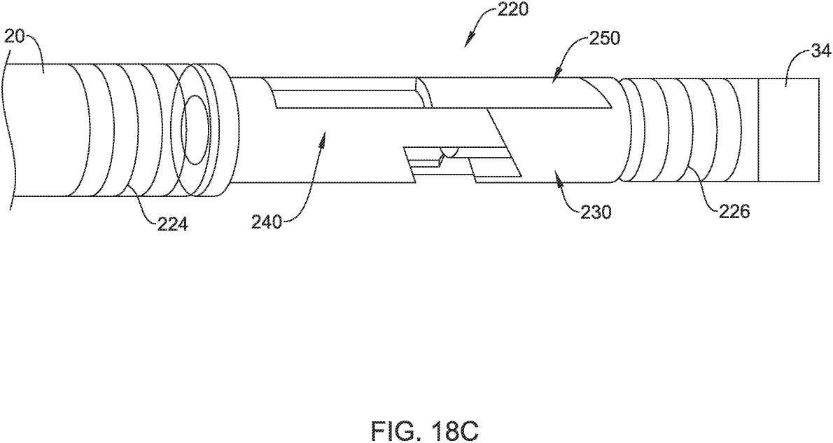

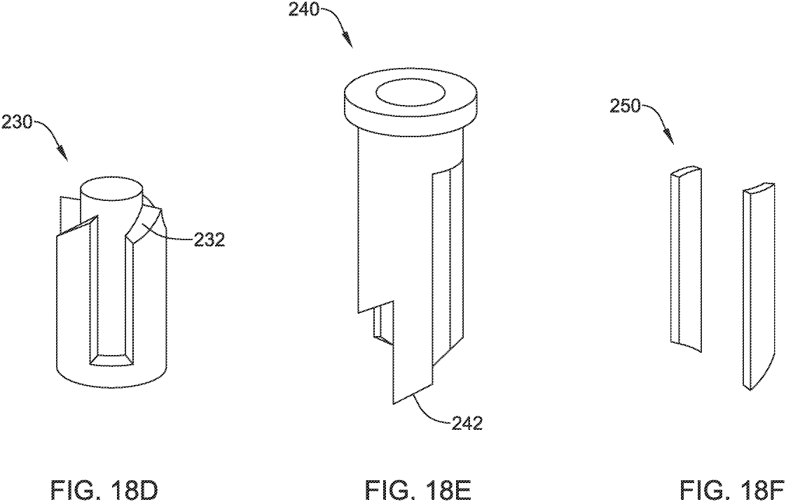

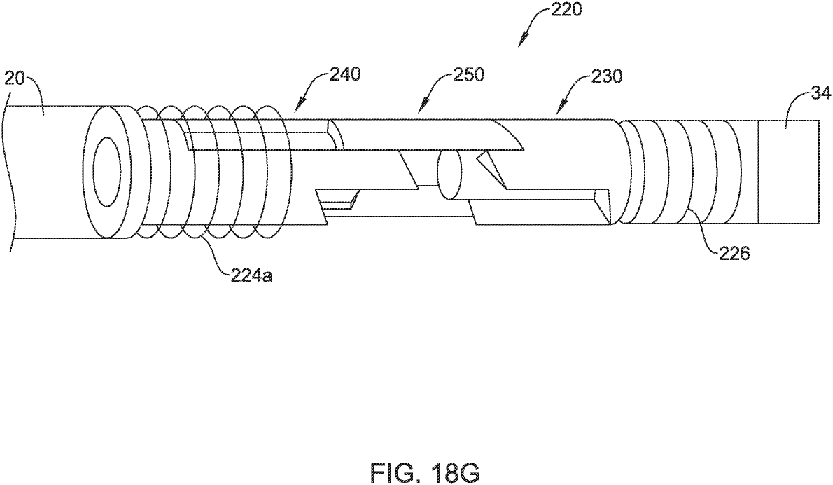

FIGS. 18A and 18B are schematic side views of an illustrative catheter 218 shown disposed within a blood vessel 60 that is defined by a vessel wall 66. A vascular asymmetric or eccentric lesion 68, which may for example be a calcified lesion, is formed within the vessel wall 66. In some cases, the lesion 68 may only extend partially circumferentially around the vessel 60. In some cases, the lesion 68 may include a single lesion 68. While not shown, in some cases there may be two or more distinct lesions 68 formed within the vessel wall 66.

The catheter 218 includes the inflatable balloon 24, as well as a plurality of traction elements 26 that are secured relative to the outer surface 28 of the inflatable balloon 24. Urging the traction elements 26 in an outward direction, such as by inflating the inflatable balloon 24, may cause one or more of the traction elements 26 to engage and stretch the lesion 68. In some cases, one or more of the traction elements 26 may crack the lesion 68. The inflatable balloon 24 includes a proximal waist 34 and a distal waist 35. The catheter 218 includes a guidewire lumen 30 that extends through the inflatable balloon 24. The guidewire lumen 30 may extend proximally to a proximal guidewire port that is disposed within the hub located in the proximal region 12 (FIG. 1), or the guidewire lumen 30 may extend proximally to a proximal guidewire port that is located just proximal of the proximal waist 34.

The catheter 218 includes a rotation mechanism 220 that is illustrated as being disposed just proximal of the proximal waist 34. In some cases, the rotation mechanism 220 may be adapted to permit relative rotation between the elongate shaft 20 and the inflatable balloon 24. In some instances, for example, the elongate shaft 20 may include an outer tubular member that terminates distally at the mechanism 220 while an inner tubular member forming an inflation lumen and/or a guidewire lumen may extend through the rotation mechanism 220 and/or distally beyond the rotation mechanism 220 and to the inflatable balloon 24.

In some cases, the rotation mechanism 220 may be actuated by applying a distally directed force to the elongate shaft 20 in a distal direction indicated by an arrow 222 while the balloon 24 is inflated within the blood vessel 60. Thus, the elongate shaft 20 may be actuated distally relative to the balloon 24 to actuate the rotation mechanism 220. It will be appreciated that while the rotation mechanism 200 (FIGS. 17A-7C) was actuated by pulling proximally on the elongate shaft 20, the rotation mechanism 220 may be actuated by pushing distally on the elongate shaft 20. In FIG. 18A, the inflatable balloon 24 may be seen as being in a first rotational orientation, as indicated by the relative position of the traction elements 26a, 26b and 26c. The rotation mechanism 220 may thus be configured to rotate the balloon 24 and associated traction elements 26 relative to the elongate shaft 20. In FIG. 18B, it can be seen that the inflatable balloon 24 has been rotated to a second rotational orientation, as indicated by the relative position of the traction elements 26b and 26c and now traction element 26d is visible while the traction element 26a is no longer visible. Pushing on the elongate shaft 20 has caused the inflatable balloon 24 and associated traction elements 26 to rotate in a direction indicated by the arrow 212.

FIGS. 18C through 18G provide some details regarding one exemplary configuration of the rotation mechanism 220. In some instances, the rotation mechanism 220 may be considered as being similar to the mechanism used in a click-to-open ball point pen. In some instances, additional details regarding the mechanism 220 may be found in US 3,205,863, which is incorporated by reference herein. It will be appreciated that the mechanism 220 may be modified from what is shown in US 3,205,863 in order to accommodate one or more tubular members extending through and/or from the rotation mechanism 220 in order to provide an inflation lumen and/or a guidewire lumen extending therethrough.

FIG. 18C shows a portion of the catheter 218 with the rotation mechanism 220 shown in its retracted position while FIG. 18G shows the mechanism 220 in an extended position. It will be appreciated that the mechanism 220 undergoes rotation in moving from the retracted position to the extended position. The mechanism 220 includes a cam body 230 that may be operably coupled with or otherwise secured relative to the proximal waist 34 of the inflatable balloon 24 and a plunger 240 that may be operably coupled with or otherwise secured relative to the elongate shaft 20. The plunger 240 may be adapted to translate longitudinally as the elongate shaft 20 translates longitudinally. The cam body 230 may be adapted to both rotate and translate longitudinally as the elongate shaft 20 translates longitudinally. A spring 224 may serve to hold the plunger 240 against the cam body 230 and/or a spring 226 may serve to hold the cam body 230 against the plunger 240.

As seen for example in FIG. 18D, the cam body 230 may include several cam surfaces, including a cam surface 232. As seen for example in FIG. 18E, the plunger 240 may include several cam surfaces including a cam surface 242 that releasably interacts with the cam surfaces including the cam surface 232 of the cam body 230. Stop members 250, which may be considered as being molded into the mechanism 220, may interact with the plunger 240 and prevent rotation of the plunger 240. As an axial force is applied to the elongate shaft 20, and hence to the plunger 240, the plunger 240 may interact with the cam body 230 in a way that causes the cam body 230 to rotate. Because the cam body 230 is operably coupled with the inflatable balloon 24, axial movement of the elongate shaft 20 may be converted into rotation of the inflatable balloon 24. The cam body 230, the plunger 240 and the stop members 250 may be formed of any suitable material, such as a polymeric material.

An additional difference between FIG. 18C and FIG. 18G is that in FIG. 18C, the spring 224 extends proximally from a proximal end of the plunger 240, while in FIG. 18G, a spring 224a engages a proximal end of the plunger 240 and extends distally therefrom. While not shown, it will be appreciated that the spring 224a may engage an interior surface of the mechanism 220 in order to maintain a biasing force on the plunger 240, pushing the plunger 240 towards the cam body 230. The springs 224, 224a and 226 may take any desired form, and may be formed of any desired material.

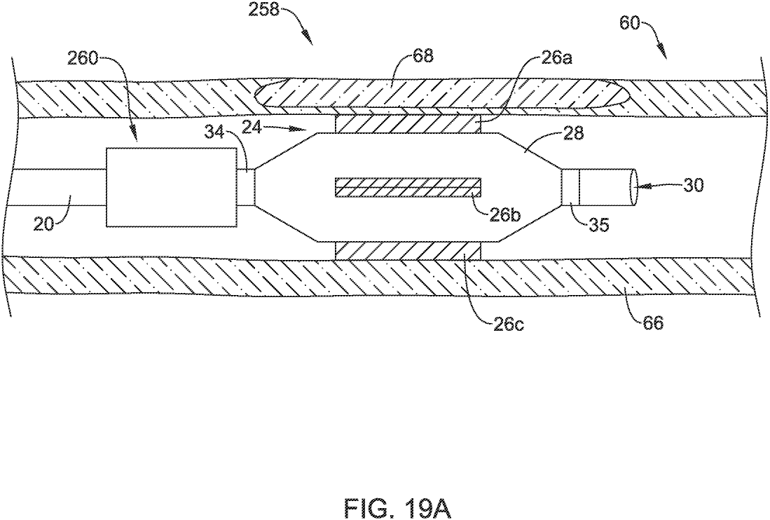

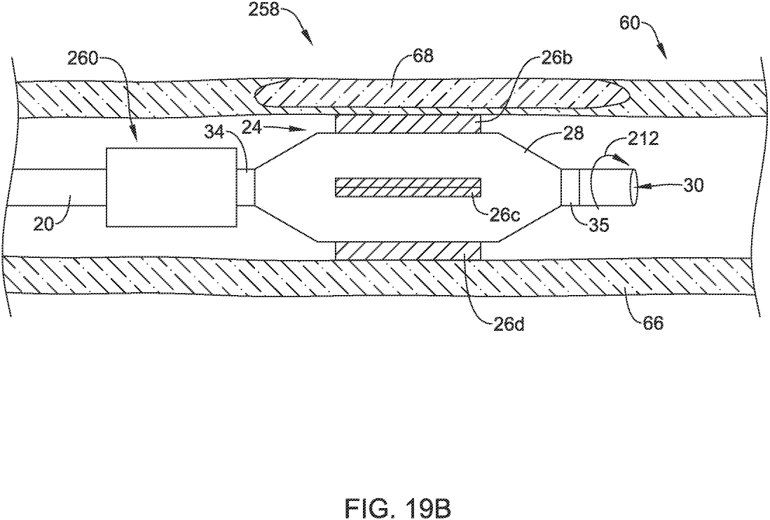

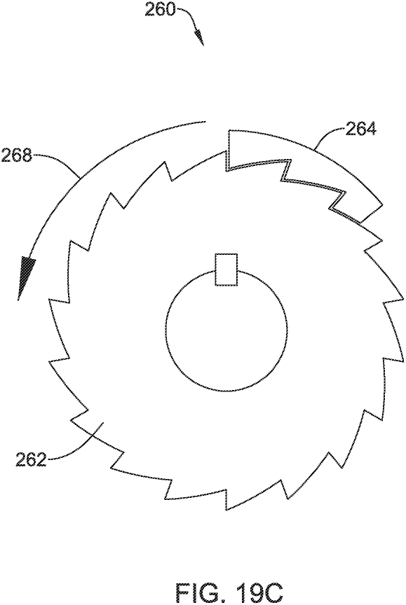

FIGS. 19A and 19B are schematic side views of an illustrative catheter 258 shown disposed within a blood vessel 60 that is defined by a vessel wall 66. A vascular asymmetric or eccentric lesion 68, which may for example be a calcified lesion, is formed within the vessel wall 66. In some cases, the lesion 68 may only extend partially circumferentially around the vessel 60. In some cases, the lesion 68 may include a single lesion 68. While not shown, in some cases there may be two or more distinct lesions 68 formed within the vessel wall 66.

The catheter 258 includes the inflatable balloon 24, as well as a plurality of traction elements 26 that are secured relative to the outer surface 28 of the inflatable balloon 24. Urging the traction elements 26 in an outward direction, such as by inflating the inflatable balloon 24, may cause one or more of the traction elements 26 to engage and stretch the lesion 68. In some cases, one or more of the traction elements 26 may crack the lesion 68. The inflatable balloon 24 includes a proximal waist 34 and a distal waist 35. The catheter 218 includes a guidewire lumen 30 that extends through the inflatable balloon 24. The guidewire lumen 30 may extend proximally to a proximal guidewire port that is disposed within the hub located in the proximal region 12 (FIG. 1), or the guidewire lumen 30 may extend proximally to a proximal guidewire port that is located just proximal of the proximal waist 34.

The catheter 258 includes a rotation mechanism 260 that is disposed just proximal of the proximal waist 34. In some cases, the rotation mechanism 260 may be adapted to permit relative rotation between the elongate shaft 20 and the inflatable balloon 24. In some instances, for example, the elongate shaft 20 may include an outer tubular member that terminates distally at the rotation mechanism 260 while an inner tubular member forming an inflation lumen and/or a guidewire lumen extends through the rotation mechanism 260 and to the inflatable balloon 24. In some cases, the rotation mechanism 260 may be considered as functioning as a ratchet, meaning that the elongate shaft 20 may be allowed to rotate in a first rotational direction relative to the inflatable balloon 24, but be inhibited from rotating in an opposing second rotational direction.

In some instances, this means that the rotation mechanism 260 may store potential energy by rotating the elongate shaft 20, for example. By deflating or at least partially deflating the inflatable balloon 24, the traction elements 26 may let go of the lesion 68, and thus allow the inflatable balloon 24 to rotate in response to releasing the potential energy built up within the rotation mechanism 260 and/or the elongate shaft 20 and/or the proximal waist 34 of the inflatable balloon 24.

In comparing FIG. 19B with FIG. 19A, it can be seen that the inflatable balloon 24 has rotated in the direction indicated by the arrow 212. In FIG. 19A, the traction elements 26a, 26b and 26c are visible, while in FIG. 19B, traction element 26a is no longer visible, traction elements 26b and 26c are visible, and now a traction element 26d can be seen.

FIG. 19C provides a schematic view of a simple example of the mechanism 260. FIG. 19C shows a toothed wheel 262 that may be considered as being coupled with the elongate shaft 20, for example. In some cases, the toothed wheel 262 may be considered as being a rachet. A pawl 264 is adapted to interact with the toothed wheel 262 in such a way that rotation of the toothed wheel 262 in a direction indicated by an arrow 268 is allowed because the pawl 264 will slip over the tops of the individual teeth on the toothed wheel 262. The pawl 264 may be considered as being coupled with the proximal waist 34 of the inflatable balloon 24, for example. If rotation of the toothed wheel 262 is attempted in a direction opposite that indicated by the arrow 268, the pawl 264 will engage the closest tooth on the toothed wheel 262 as a result of a biasing force applied to the pawl 264, such as by a spring (not shown), and rotation will be prevented.

Thus, the rachet (e.g., toothed wheel 262) may work together with the pawl 264 to enable relative rotation in a first direction of the catheter shaft 20 relative to the balloon 24 while preventing relative rotation therebetween in an opposing direction. It will be appreciated that in some cases, the pawl 264 may instead be a second toothed wheel, and may be moved between allowing rotation in a first direction (but not a second direction) and allowing rotating in the second direction (but not in the first direction). In some cases, a ratchet mechanism such as the rotation mechanism 260 may also include additional features that replace the spring biasing element, and allow switching between which direction rotation is allowed and which direction rotation is prevented.

It is noted that the rotation mechanisms 200, 220, 260 are illustrated as providing controlled rotation between an elongate shaft 20 of a catheter and a balloon 24 having traction elements 26 mounted thereon, in some instances, the rotation mechanism 200, 220, 260 may be incorporated with a cage (such as any of the cage-like structures disclosed herein), providing or defining traction elements, that surrounds or is otherwise no fixed relative to the balloon 24. Thus the rotation mechanism 200, 220, 260 may be configured to rotate the cage and associated traction elements relative to the catheter shaft 20 and the balloon 24.

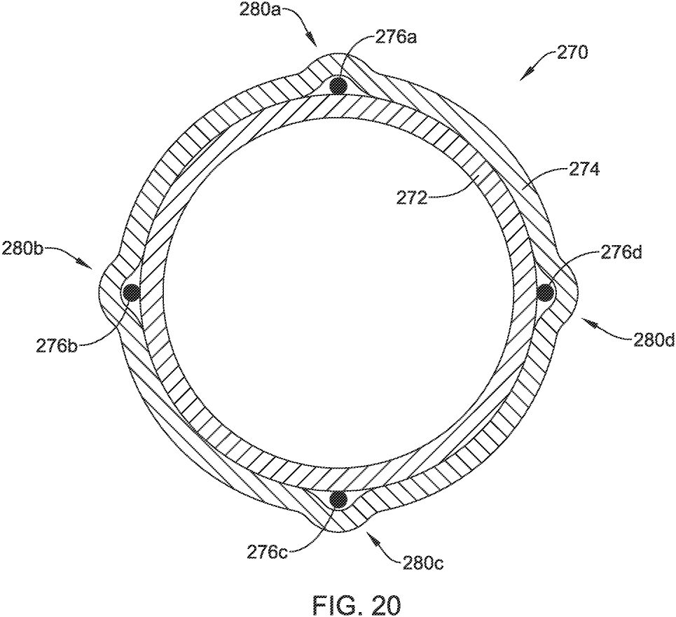

FIG. 20 is a schematic cross-sectional view of an inflatable balloon 270, showing an example of how traction elements may be added to an inflatable balloon 270. In some cases, as shown for example in FIG. 1A, the traction elements 26 may be adhesively secured relative to the outer surface 28 of the inflatable balloon 24. In some cases, as shown in FIG. 20, the inflatable balloon 270 may include an inner polymeric layer 272 and an outer polymeric layer 274. The placement of the traction elements 26 between polymeric layers of the balloon 270 may result in a plurality or raised ridges extending along the exterior surface of the balloon 270. While only two polymeric layers 272 and 274 are shown, it will be appreciated that in some cases the inflatable balloon 270 may include one or more additional layers. The raised ridges may define traction elements 280 extending along the exterior surface of the balloon 270. As shown, a total of four traction elements 280, individually labeled as 280a, 280b, 280c and 280d, are formed by disposing elongate elements 276, individually labeled as 276a, 276b, 276c and 276d, between the inner polymeric layer 272 and the outer polymeric layer 274. The elongate elements 276 form raised ridges that create the traction elements 280.

As shown, each of the elongate elements 276 have a round cross-sectional profile. In some cases, each of the elongate elements 276 may have a triangular cross-sectional profile, for example. In some cases, each of the elongate elements 276 may have a square cross-sectional profile, a rectilinear cross-sectional profile, or other polygonal cross-sectional profile. Other profiles are also contemplated. The elongate elements 276 may be polymeric, for example. In some cases, the elongate elements 276 may be metallic.

The catheter 10, and various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. A method of treating a lesion within a blood vessel, the method comprising:
advancing a catheter through the vessel to a treatment site proximate the lesion, the catheter including a torqueable shaft extending from a hub fixedly secured to a proximal end region of the torqueable shaft to an inflatable balloon fixedly secured to a distal end region of the torqueable shaft, the catheter including a plurality of traction elements disposed about the inflatable balloon, wherein the inflatable balloon is adapted to urge the plurality of traction elements radially outwardly when the inflatable balloon is inflated;
thereafter, inflating the inflatable balloon to urge one or more of the plurality of traction elements radially outwardly into contact with the lesion;
thereafter, deflating the inflatable balloon;
thereafter, rotating the hub of the catheter in order to achieve a corresponding rotation of the balloon of the catheter relative to the treatment site; and
thereafter, re-inflating the inflatable balloon to urge one or more of the plurality of traction elements radially outwardly into contact with the lesion;
wherein at least two of the plurality of traction elements concurrently engage the lesion during the step of inflating the inflatable balloon and/or during the step of re-inflating the inflatable balloon.

2. The method of claim 1, wherein for a given angle of rotation of the hub of the catheter, the balloon of the catheter rotates in the same direction an angle or rotation that is within twenty percent of the given angle of rotation.

3. The method of claim 1, wherein for a given angle of rotation of the hub of the catheter, the balloon of the catheter rotates in the same direction an angle of rotation that is within ten percent of the given angle of rotation.

4. The method of claim 1, wherein urging the at least two of the plurality of traction elements radially outwardly into contact with the lesion causes at least a portion of the lesion to stretch or crack.

5. The method of claim 1, wherein the plurality of traction elements are a plurality of cutting members circumferentially spaced around an outer surface of the inflatable balloon.

6. The method of claim 1, wherein the torqueable shaft includes a braid or coil extending from the hub to a proximal waist of the balloon.

7. The method of claim 1, wherein the lesion is an eccentric lesion extending less than 180 degrees around the vessel, wherein the lesion is cracked during the step of inflating the inflatable balloon and/or the step of re-inflating the inflatable balloon.

8. A method of treating a lesion within a blood vessel, the method comprising:
advancing a catheter to a treatment site proximate a lesion, the catheter including a proximal region including a hub and a distal region including a plurality of traction elements and an inflatable balloon adapted to urge the plurality of traction elements radially outwardly when the inflatable balloon is inflated, the catheter including a mechanism disposed between the proximal region and the distal region, the mechanism adapted to convert axial movement of the proximal region into rotation of the distal region;
thereafter, inflating the inflatable balloon to urge at least one of the plurality of traction elements radially outwardly into contact with the lesion with the distal region at a first rotational orientation;
thereafter, causing axial movement of the proximal region, thereby causing the mechanism to twist the distal region, temporarily placing energy into the distal region;
thereafter, deflating the inflatable balloon in order to allow the distal region to untwist, thereby rotating the distal region to a second rotational orientation different form the first rotational orientation;
thereafter, re-inflating the inflatable balloon to urge at least one of the plurality of traction elements radially outwardly into contact with the lesion;
wherein at least two of the plurality of traction elements concurrently engage the lesion during the step of inflating the inflatable balloon and/or during the step of re-inflating the inflatable balloon.

9. The method of claim 8, wherein the mechanism is adapted such that pushing on the proximal region of the catheter causes the mechanism to twist the distal region.

10. The method of claim 8, wherein the mechanism is adapted such that pulling on the proximal region of the catheter causes the mechanism to twist the distal region.

11. The method of claim 8, wherein the catheter includes a shaft extending from the hub to the inflatable balloon.

12. The method of claim 8, wherein the lesion is an eccentric lesion extending less than 180 degrees around the vessel, wherein the lesion is cracked during the step of inflating the inflatable balloon and/or the step of re-inflating the inflatable balloon.

13. A device for treating an eccentric lesion within a blood vessel, the device comprising:
an elongate shaft including a distal region and a proximal region, the elongate shaft configured to provide torque transmission between the proximal region and the distal region;
a hub fixedly secured to the proximal region of the elongate shaft;
an inflatable balloon fixedly secured to the distal region of the elongate shaft;
a plurality of traction elements disposed on the inflatable balloon such that the plurality of traction elements are urged radially outwardly when the inflatable balloon is inflated such that at least two of the plurality of traction elements concurrently engage the eccentric lesion within the blood vessel.

14. The device of claim 13, wherein the elongate shaft is configured to provide, for a given angle of rotation of the hub, a rotation of the balloon in the same direction an angle of rotation that is within twenty percent of the given angle of rotation of the hub.

15. The device of claim 13, wherein the elongate shaft comprises one or more torque-transmission elements extending within the elongate shaft.

16. The device of claim 13, wherein the one or more torque-transmission elements comprises a reinforcing member extending within a tubular member of the elongate shaft.

17. The device of claim 16, wherein the reinforcing member comprises at least one of a braid and a coil.

18. The device of claim 17, wherein the braid or coil extends from the hub to a proximal waist of the balloon.

19. The device of claim 13, wherein the plurality of traction elements are configured such that urging the plurality of traction elements radially outwardly into contact with the lesion causes at least a portion of the lesion to stretch or crack.

20. The device of claim 13, wherein the plurality of traction elements are a plurality of cutting members circumferentially spaced around an outer surface of the inflatable balloon.
